(19) United States
(12) Patent Application Publication
(10) Pub. No.: US 2025/0262169 A1
(43) Pub. Date: Aug. 21, 2025

(54) METHODS OF TREATING COENZYME Q10 DEFICIENCY

(71) Applicant: BPGbio, Inc., Framingham, MA (US)

(72) Inventors: Vijay Ramakrishnan Modur, Acton, MA (US); Niven Rajin Narain, Cambridge, MA (US)

(21) Appl. No.: 19/056,653

(22) Filed: Feb. 18, 2025

Related U.S. Application Data
(60) Provisional application No. 63/554,899, filed on Feb. 16, 2024.
(60) Provisional application No. 63/554,900, filed on Feb. 16, 2024.

(51) Int. Cl. A61K 31/122; A61K 9/00; A61K 9/107; A61P 3/02

(52) U.S. Cl. CPC A61K 31/122; A61K 9/0029; A61K 9/0053; A61K 9/1075; A61P 3/02

(57) ABSTRACT

Provided herein are methods of treating primary Coenzyme Q10 deficiency in a subject, comprising administering to the subject a highly bioavailable composition comprising Coenzyme Q10. The methods result in plasma concentrations of Coenzyme Q10 significantly greater than normal levels in a subject, and far greater than can be achieved with over the counter oral Coenzyme Q10 supplements.

### RELATED APPLICATIONS

This application claims the benefit of priority to U.S. Provisional Application No. 63/554,899, filed on Feb. 16, 2024, and U.S. Provisional Application No. 63/554,900, filed on Feb. 16, 2024. The entire contents of each of the foregoing applications are incorporated herein by reference.

### BACKGROUND

Coenzyme Q10 (CoQ10) is a popular nutritional supplement and can be found in capsule form in nutritional stores, health food stores, pharmacies, and the like, as a vitamin-like supplement to help improve mitochondrial function and provide benefits through the antioxidant properties of ubiquinol, the reduced form of CoQ10. CoQ10 is found throughout most tissues of the human body and the tissues of other mammals and is concentrated in the mitochondria.

CoQ10 has a number of vital functions in all cells, both mitochondrial and extra-mitochondrial. In addition to its key role in mitochondrial oxidative phosphorylation, CoQ10 serves as a lipid soluble antioxidant and plays an important role in fatty acid beta-oxidation and pyrimidine and lysosomal metabolism, as well as directly mediating the expression of a number of genes, including those involved in inflammation. A deficiency in CoQ10 has been implicated in the pathogenesis of a wide range of disorders.

CoQ10 deficiency is broadly divided into primary and secondary types. Primary CoQ10 deficiency results from mutations in genes involved in the CoQ10 biosynthetic pathway, whereas secondary deficiencies can occur as result of other diseases or certain pharmacotherapies. In human, at least 10 genes are required for the biosynthesis of functional CoQ10, a mutation in any one of which can result in a deficit in CoQ10 status.

The severity, combination of signs and symptoms, and age of onset of primary coenzyme Q10 deficiency vary widely. In the most severe cases, the condition becomes apparent in infancy and causes severe brain dysfunction combined with muscle weakness (encephalomyopathy) and the failure of other body systems. These problems can be life-threatening. Other neurological abnormalities that can occur in primary coenzyme Q10 deficiency include seizures, intellectual disability, poor muscle tone (hypotonia), involuntary muscle contractions (dystonia), progressive muscle stiffness (spasticity), abnormal eye movements (nystagmus), vision loss caused by degeneration (atrophy) of the optic nerves or breakdown of the light-sensing tissue at the back of the eyes (retinopathy), and sensorineural hearing loss (which is caused by abnormalities in the inner ear). The neurological problems gradually get worse unless treated with coenzyme Q10 supplementation.

Because of the extreme hydrophobicity and relatively large size of CoQ10, absorption and effective tissue distribution of orally administered CoQ10 has been reported to be poor. Thus, improved methods for the treatment of CoQ10 deficiency remain desirable.

### SUMMARY OF THE INVENTION

The present invention is based, at least in part, on the unexpected and surprising discovery that administration of a highly bioavailable CoQ10 composition results in a significant increase in the plasma level of CoQ10 in patients and in mice concomitantly greatly increases tissue levels after administration, unlike orally administered CoQ10. Specifically, the present inventors have successfully demonstrated that an about 200-fold increase in the plasma concentration of CoQ10 was achieved upon intravenous administration of the CoQ10 composition, an increase substantially greater than that reported with oral CoQ10 supplements. For example, plasma concentrations of CoQ10 in normal healthy individuals have been reported to be in the range of 0.76-1.53 μM, and were reported to be increased to 1.33-3.17 μM following oral CoQ10 supplementation (Paredes-Fuentes et al., 2020, Antioxidants, 9:979, the entire contents of which are incorporated herein by reference). The methods provided herein result in CoQ10 plasma concentrations of at least 10 UM, and including 1 mM. Accordingly, the compositions and methods provided herein are highly suitable for treating subjects suffering from primary CoQ10 deficiency.

Accordingly, in one aspect, the present invention provides a method for treating primary Coenzyme 10 (CoQ10) deficiency in a subject in need thereof, comprising administering parenterally (e.g., intravenously) to the subject a therapeutically effective amount of a composition comprising CoQ10, thereby treating the subject.

In another aspect, the present invention provides a method for treating primary Coenzyme 10 (CoQ10) deficiency in a subject in need thereof, comprising (i) administering parenterally (e.g., intravenously) to the subject a therapeutically effective amount of a composition comprising CoQ10, and (ii) administering orally a therapeutically effective amount of the composition comprising CoQ10, threby treating the subject.

In one aspect, the present invention provides a method for treating primary Coenzyme 10 (CoQ10) deficiency in a subject in need thereof, comprising administering parenterally to the subject a therapeutically effective amount of a composition comprising CoQ10, such that a plasma concentration of at least about 10 μg/mL or 0.01 mM for Co10 is achieved in the subject, thereby treating the subject.

In another aspect, the present invention provides a method for treating primary Coenzyme 10 (CoQ10) deficiency in a subject in need thereof, comprising (i) administering parenterally to the subject a therapeutically effective amount of a composition comprising CoQ10, and (ii) administering orally a therapeutically effective amount of the composition comprising CoQ10; such that a plasma concentration of at least about 10 μg/mL or 0.01 mM for Co10 is achieved in the subject, thereby treating the subject.

In some embodiments, the method further comprises selecting a subject determined as having primary CoQ10 deficiency.

In some embodiments, the subject is determined as having primary CoQ10 deficiency based on the presence of a loss of function in one or more genes selected the group consisting of PDSS1, PDSS2, COQ2, COQ4, COQ5, COQ6, COQ7, COQ8A, COQ8B, and COQ9.

In some embodiments, a plasma concentration of about 10 μg/mL to about 3000 μg/mL, or about 0.01 mM to about 3.0 mM, for CoQ10 is achieved in the subject. In some embodiments, a plasma concentration of about 10 μg/mL to about 2500 μg/mL, or about 0.01 mM to about 2.5 mM, for CoQ10 is achieved in the subject. In some embodiments, a plasma concentration of about 10 μg/mL to about 2000 μg/mL, or about 0.01 mM to about 2.0 mM, for CoQ10 is achieved in the subject.

In some embodiments, a plasma concentration of at least about 20 μg/mL or 0.02 mM for CoQ10 is achieved in the subject. In some embodiments, a plasma concentration of at least about 50 μg/mL or 0.05 mM for CoQ10 is achieved in the subject. In some embodiments, a plasma concentration of at least about 100 μg/mL or 0.1 mM for CoQ10 is achieved in the subject. In some embodiments, a plasma concentration of at least about 200 μg/mL or 0.2 mM for CoQ10 is achieved in the subject. In some embodiments, a plasma concentration of at least about 500 μg/mL or 0.5 mM for CoQ10 is achieved in the subject.

In some embodiments, the composition is formulated as a nano-dispersion. In some embodiments, the composition comprises an aqueous solution; a CoQ10 dispersed into a colloidal nano-dispersion of particles; and at least one of a dispersion stabilizing agent and an opsonization reducer; wherein the nano-dispersion of the CoQ10 is dispersed into nano-particles having a mean particle size of less than 200-nm.

In some embodiments, the dispersion stabilizing agent is selected the group consisting of pegylated castor oil, Cremphor EL, Cremophor RH 40, Pegylated vitamin E, Vitamin E TPGS, and Dimyristoylphosphatidyl choline (DMPC). In some embodiments, the dispersion stabilizing agent is DMPC.

In some embodiments, the oposonization reducer is selected from the group consisting of poloxamer and poloxamines. In some embodiments, the opsonization reducer is poloxamer 188. In some embodiments, the opsonization reducer is poloxamer 188 and the dispersion stabilizing agent is DMPC.

In some embodiments, the colloidal nano-dispersion is a suspension. In some embodiments, the colloidal nano-dispersion is an emulsion.

In some embodiments, the CoQ10 of the colloidal nano-dispersion is in a crystalline form. In some embodiments, the CoQ10 of the colloidal nano-dispersion is in a super-cooled melt form.

In some embodiments, the composition comprising CoQ10 has a weight-per-volume of the CoQ10, DMPC and poloxamer of 4%, 3% and 1.5%, respectively. In some embodiments, the composition comprising CoQ10 has a weight-per-volume of the CoQ10, DMPC and poloxamer 8%, 6% and 3%, respectively.

In some embodiments, the size of the nano-dispersion particles is between 10-nm and 200-nm. In some embodiments, the size of the nano-dispersion particles is between 10-nm and 100-nm. In some embodiments, the size of the nano-dispersion particles is between 35-nm and 40-nm.

In another aspect, the present invention provides a method for treating primary Coenzyme Q10 (CoQ10) deficiency in a subject in need thereof, comprising administering parenterally to the subject a therapeutically effective amount of a composition comprising CoQ10, wherein the composition comprises an aqueous solution; a CoQ10 dispersed into a colloidal nano-dispersion of particles; and at least one of a dispersion stabilizing agent and an opsonization reducer; wherein the nano-dispersion of the CoQ10 is dispersed into nano-particles having a mean particle size of less than 200-nm, thereby treating the subject.

In another aspect, the present invention provides a method for treating primary Coenzyme Q10 (CoQ10) deficiency in a subject in need thereof, comprising (i) administering parenterally to the subject a therapeutically effective amount of a composition comprising CoQ10, and (ii) administering orally a therapeutically effective amount of the composition comprising CoQ10; wherein the composition comprises an aqueous solution; a CoQ10 dispersed into a colloidal nano-dispersion of particles; and at least one of a dispersion stabilizing agent and an opsonization reducer; wherein the nano-dispersion of the CoQ10 is dispersed into nano-particles having a mean particle size of less than 200-nm, thereby treating the subject.

In some embodiments, the method further comprises selecting a subject determined as having primary CoQ10 deficiency. In some embodiments, the subject is determined as having primary CoQ10 deficiency based on the presence of a loss of function in one or more genes selected the group consisting of PDSS1, PDSS2, COQ2, COQ4, COQ5, COQ6, COQ7, COQ8A, COQ8B, and COQ9.

In some embodiments, a plasma concentration of about 10 μg/mL to about 3000 μg/mL, or about 0.01 mM to about 3.0 mM, for CoQ10 is achieved in the subject. In some embodiments, a plasma concentration of about 10 μg/mL to about 2500 μg/mL, or about 0.01 mM to about 2.5 mM, for CoQ10 is achieved in the subject. In some embodiments, a plasma concentration of about 10 μg/mL to about 2000 μg/mL, or about 0.01 mM to about 2.0 mM, for CoQ10 is achieved in the subject.

In some embodiments, a plasma concentration of at least about 20 μg/mL or 0.02 mM for CoQ10 is achieved in the subject. In some embodiments, a plasma concentration of at least about 50 μg/mL or 0.05 mM for CoQ10 is achieved in the subject. In some embodiments, a plasma concentration of at least about 100 μg/mL or 0.1 mM for CoQ10 is achieved in the subject. In some embodiments, a plasma concentration of at least about 200 μg/mL or 0.2 mM for CoQ10 is achieved in the subject. In some embodiments, a plasma concentration of at least about 500 μg/mL or 0.5 mM for CoQ10 is achieved in the subject.

In some embodiments, the oposonization reducer is selected from the group consisting of poloxamer and poloxamines. In some embodiments, the opsonization reducer is poloxamer 188. In some embodiments, the opsonization reducer is poloxamer 188 and the dispersion stabilizing agent is DMPC.

In some embodiments, the colloidal nano-dispersion is a suspension. In some embodiments, the colloidal nano-dispersion is an emulsion.

In some embodiments, the CoQ10 of the colloidal nano-dispersion is in a crystalline form. In some embodiments, the CoQ10 of the colloidal nano-dispersion is in a super-cooled melt form.

In some embodiments, the composition comprising CoQ10 has a weight-per-volume of the CoQ10, DMPC and poloxamer of 4%, 3% and 1.5%, respectively. In some embodiments, the composition comprising CoQ10 has a weight-per-volume of the CoQ10, DMPC and poloxamer 8%, 6% and 3%, respectively.

In some embodiments, the size of the nano-dispersion particles is between 10-nm and 200-nm. In some embodiments, the size of the nano-dispersion particles is between 10-nm and 100-nm. In some embodiments, the size of the nano-dispersion particles is between 35-nm and 40-nm.

In some embodiments, the composition is administered by intravenous infusion.

In some embodiments, the composition is administered once per week.

In some embodiments, the composition is administered twice per week.

In some embodiments, the composition is administered intravenously once per week. In some embodiments, the composition is administered intravenously twice per week.

In some embodiments, the composition is administered at a dose of between about 5 mg/kg to about 350 mg/kg of CoQ10 per week. In some embodiments, the composition is administered at a dose of between about 10 mg/kg to about 100 mg/kg of CoQ10 per week. In some embodiments, the composition is administered at a dose of between about 25 mg/kg and about 100 mg/kg of CoQ10 per week. In some embodiments, the composition is administered at a dose of between about 25 mg/kg to about 75 mg/kg of CoQ10 per week.

In some embodiments, the composition is administered at a dose of about 5 mg/kg, about 10 mg/kg, about 15 mg/kg, about 20 mg/kg, about 25 mg/kg, about 50 mg/kg, about 100 mg/kg, about 150 mg/kg, about 200 mg/kg, about 250 mg/kg, about 300 mg/kg, or about 350 mg/kg of CoQ10 per week. In some embodiments, the composition is administered parentally (e.g., intravenously) at a dose of about 50 mg/kg per week.

In some embodiments, the composition is administered parentally (e.g., intravenously) at a dose of between about 5 mg/kg to about 350 mg/kg of CoQ10 per week. In some embodiments, the composition is administered parentally (e.g., intravenously) at a dose of between about 10 mg/kg to about 100 mg/kg of CoQ10 per week. In some embodiments, the composition is administered parentally (e.g., intravenously) at a dose of between about 25 mg/kg and about 100 mg/kg of CoQ10 per week. In some embodiments, the composition is administered parentally (e.g., intravenously) at a dose of between about 25 mg/kg to about 75 mg/kg of CoQ10 per week.

In some embodiments, the composition is administered parentally (e.g., intravenously) at a dose of about 5 mg/kg, about 10 mg/kg, about 15 mg/kg, about 20 mg/kg, about 25 mg/kg, about 50 mg/kg, about 100 mg/kg, about 150 mg/kg, about 200 mg/kg, about 250 mg/kg, about 300 mg/kg, or about 350 mg/kg of CoQ10 per week. In some embodiments, the composition is administered parentally (e.g., intravenously) at a dose of about 50 mg/kg per week.

In some embodiments, the composition is administered orally according to an administration schedule selected from once per week, twice per week, three times per week, four times per week, five times per week, six times per week, or once per day.

In some embodiments, the composition is administered orally at a dose of between about 5 mg/kg to about 5000 mg/kg of CoQ10 per day. In some embodiments, the composition is administered orally at a dose of between about 5 mg/kg to about 3500 mg/kg of CoQ10 per day. In some embodiments, the composition is administered orally at a dose of between about 10 mg/kg to about 3500 mg/kg of CoQ10 per day. In some embodiments, the composition is administered orally at a dose of between about 20 mg/kg to about 2000 mg/kg of CoQ10 per day. In some embodiments, the composition is administered orally at a dose of between about 20 mg/kg to about 1000 mg/kg of CoQ10 per day. In some embodiments, the composition is administered orally at a dose of between about 10 mg/kg to about 500 mg/kg of CoQ10 per day. In some embodiments, the composition is administered orally at a dose of between about 5 mg/kg to about 100 mg/kg of CoQ10 per day. In some embodiments, the composition is administered orally at a dose of between about 100 mg to about 5000 mg of CoQ10 per day. In some embodiments, the composition is administered orally at a dose of between about 500 mg to about 5000 mg of CoQ10 per day. In some embodiments, the composition is administered orally at a dose of between about 1000 mg to about 5000 mg of CoQ10 per day. In some embodiments, the composition is administered orally at a dose of between about 1500 mg to about 5000 mg of CoQ10 per day. In some embodiments, the composition is administered orally at a dose of between about 2000 mg to about 5000 mg of CoQ10 per day. In some embodiments, the composition is administered orally at a dose of between about 3000 mg to about 5000 mg of CoQ10 per day. In some embodiments, the composition is administered orally at a dose of between about 3000 mg to about 3500 mg of CoQ10 per day.

In some embodiments, the composition is administered orally at a dose of about 5 mg/kg, about 10 mg/kg, about 15 mg/kg, about 20 mg/kg, about 25 mg/kg, about 50 mg/kg, about 100 mg/kg of CoQ10, about 200 mg/kg, about 500 mg/kg, about 1000 mg/kg, about 1500 mg/kg or about 2000 mg/kg per day.

In some embodiments, the composition is administered orally at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4500 mg, or about 5000 mg, of CoQ10 per day.

In some embodiments, the methods further comprise administering a CoQ10 supplement orally to the subject. In some embodiments, the CoQ10 supplement is administered orally at a dose of between about 10 mg/kg and about 100 mg/kg per day. In some embodiments, the CoQ10 supplement is administered orally at a dose of between about 10 mg/kg and about 50 mg/kg per day. In some embodiments, the CoQ10 supplement is administered orally at a dose of about 30 mg/kg per day.

In some embodiments, the subject is a human subject.

In some embodiments, administration of the composition comprising CoQ10 results in a concentration of CoQ10 in the brain, heart, kidney, and/or muscle tissues that is at least 1.1-fold, e.g., at least 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 30-fold, 40-fold, 50-fold, 100-fold, or 150 fold, greater than the concentration of CoQ10 in the brain, heart, kidney, and/or muscle tissues measured in the subject prior to administration of the composition.

In some embodiments, administration of the composition comprising CoQ10 restores the level of CoQ10 in the subject having primary CoQ10 deficiency to a normal physiological level or above the physiological level in the brain, heart, kidney and/or muscle tissues of the subject.

In some embodiments, administration of the composition comprising CoQ10 results in a change in the levels of lactate, succinate and/or citrate in the brain, heart, kidney and/or muscle tissues of the subject.

In some embodiments, administration of the composition comprising CoQ10 increases the plasma concentration of one or more metabolites of quinone metabolism.

In some embodiments, the one or more metabolites of quinone metabolism is selected from the group consisting of Coenzyme Q1, Coenzyme Q2, Coenzyme Q4, phylloquinone, menaquinone, menadione, 1,2-naphthoquinone, D-alpha-tocopherylquinone, p-Benzoquinone, duroquinone, idebenone, 2-methoxy-1,4-naphthoquinone, 2,6-dimethoxy-1,4-benzoquinone, adrenochrome, 1,8-dihydroxyanthraquinone, chrysophanol, thymoquinone, 2,6-di-tert-butyl-1,4-benzoquinone, emodin, and pyrroloquinoline quinone. In some embodiments, the one or more metabolites of quinone metabolism is selected from the group consisting of menadione, duroquinone, and 1,8-dihydroxyanthraquinone.

In some embodiments, administration of the composition comprising CoQ10 results in a decrease in a Scale for the Assessment and Rating of Ataxia (SARA) score by at least 1, e.g., at least 1.1, 1.2, 1.3, 1.4, 1.5, 1.6, 1.7, 1.8, 1.9, 2, 2.5, or 3, as compared to a SARA score measured in the subject prior to administration of the composition.

In some embodiments, administration of the composition comprising CoQ10 results in a decrease in the time required to complete a 9-Hole Peg Test (9HPT) by at least 1, e.g., at least 2, 3, 4, 5, 6, 7, 8, 9, or 10 seconds, as compared to the time required to complete the test by the subject prior to administration of the composition.

In some embodiments, administration of the composition comprising CoQ10 results in a decrease in a Friedreich's Ataxia Rating Scale-Activities of Daily Living (FARS-ADL) score by at least 1, e.g., at least 1.5, 2, 2.5, 3, 3.5, 4, 4.5, 5, 5.5, or 6, as compared to a FARS-ADL score measured in the subject prior to administration of the composition.

In some embodiments, administration of the composition comprising CoQ10 results in an increase in a Patient Global Impression of Change (PGIC) score by at least 1, 2, or 3, as compared to a PGIC score measured in the subject prior to administration of the composition.

### BRIEF DESCRIPTION OF THE FIGURES

FIG. 1 depicts the plasma levels of CoQ in patients administered intravenously with two 72-hour weekly doses of BPM31510.

FIGS. 2A-2D depict the oxidized CoQ10 levels in mice after 2 weeks of oral CoQ10 treatment, or treatment with BPM31510 intraperitoneally (IP) at a dose of 10 mg/kg or 50 mg/kg twice a day (BID). FIG. 2A depicts the oxidized CoQ10 levels in cerebellum. FIG. 2B depicts the oxidized CoQ10 levels in kidney. FIG. 2C depicts the oxidized CoQ10 levels in heart. FIG. 2D depicts the oxidized CoQ10 levels in muscle.

FIGS. 3A-3B depict the PABA Treatment and BPM31510 exposure on oxidized CoQ10 levels in SH-SY5Y cell cultures. FIG. 3A depicts that SH-SY5Y cultures were treated with either vehicle or 1 mM PABA for 5 days. CoQ10 levels are expressed as μg per 1 million cells. (**** p<0.0001; t-test). FIG. 3B depicts that SH-SY5Y were treated for 5 days with 1 mM PABA and then treated for an additional 5 days with either 1 mM PABA alone or PABA plus the indicated amounts of BPM31510. (**** p<0.0001; one-way ANOVA; Dunnett's multiple comparison test; samples compared to 1 mM PABA treatment alone). Data were from three independent experiments containing triplicate samples for each condition.

FIG. 4 depicts the effect of BPM31510 on ATP generation. * p<0.05, *** p<0.001 (one-way ANOVA; Dunnett's multiple comparison test; samples compared to 1 mM PABA treatment alone); Error bars represent SEM.

FIG. 5 depicts that BPM31510 decreases mitochondrial ROS to normal levels. * p<0.05, *** p<0.001 (one-way ANOVA; Dunnett's multiple comparison test; samples compared to 1 mM PABA treatment alone); Error bars represent SEM; MFI=mean fluorescence intensity.

FIG. 6 depicts the ATP levels in CoQ10 deficient patient fibroblasts, PDSS2 and CoQ2 mutant fibroblast cell lines, treated with 5 μM BPM31510 for 24 hours.

FIG. 7 depicts the levels of CoQ10 in a CoQ2 deficient fibroblast cell line and a control fibroblast cell line treated with BPM31510, or a control CoQ10 formulation for 24 hours or 1 week.

FIG. 8 depicts the levels of CoQ10 in gastrocnemius muscle, brain, heart, and kidney tissues from mice having primary CoQ10 deficiency (CoQ4 KI deficient mice), or wild type mice, upon receiving a BPM31510 treatment, or a vehicle control, every other day for two weeks.

FIG. 9 are images from a spatial omics analysis depicting the CoQ10 distribution in the heart of wild type and CoQ4 KI deficient mice treated with BPM31510.

FIG. 10 depicts the levels of CoQ10 and CoQ9 in vascular enriched locations (ROI1) and muscle tissue enriched regions (ROI 2) in wild type and CoQ4 KI mutant mice treated with BPM31510.

FIG. 11 are images from a spatial omics analysis depicting the CoQ10 distribution in the brain regions of wild type and CoQ4 KI deficient mice treated with BPM31510.

FIG. 12 depicts the levels of CoQ10 and CoQ9 in cerebellum of wild type and CoQ4 KI mutant mice treated with BPM31510.

FIG. 13 depicts the levels of CoQ10 and CoQ9 in white matter enriched regions (RO1,, grey matter enriched regions (RO2), and vascular enriched locations (ROI3) in wild type and CoQ4 KI mutant mice treated with BPM31510.

FIG. 14 are images from a spatial omics analysis depicting the CoQ10 distribution in the kidney regions of wild type and CoQ4 KI deficient mice treated with BPM31510.

FIG. 15 depicts the levels of CoQ10 and CoQ9 in the outer cortex (RO1), inner cortex (RO2), medulla (RO3), pelvis (RO4) and vascular enriched regions (RO5) of the kidney in wild type and CoQ4 KI mutant mice treated with BPM31510.

FIG. 16 depicts the significant improvement in the clinical-reported outcome (SARA score) for patients receiving the BPM31510 treatment.

FIG. 17 depicts the significant improvement in the performance outcome (9-Hole Peg-Test) for patients receiving the BPM31510 treatment.

FIG. 18 depicts the significant improvement in patient-reported outcomes (FARS-ADL and PGIC) for patients receiving the BPM31510 treatment.

FIG. 19 depicts the significant improvement in observer-reported outcomes for patients receiving the BPM31510 treatment.

FIG. 20 depicts the significant improvement in gait and balance, dexterity, speech and cognition for patients receiving the BPM31510 treatment.

FIG. 21 depicts the significant improvement in goal attainment for patients receiving the BPM31510 treatment.

### DETAILED DESCRIPTION OF THE INVENTION

The present invention is based, at least in part, on the unexpected and surprising discovery that administration of a highly bioavailable CoQ10 composition results in a significant increase in the plasma level of CoQ10 in patients. Specifically, the present inventors have successfully demonstrated that an about 200-fold increase in the plasma concentration of CoQ10 was achieved upon administration, e.g., intravenous administration and/or oral administration, of the CoQ10 composition over normal plasma levels.

Accordingly, the present invention provides methods for treating primary CoQ10 deficiency in a subject in need thereof, by administering, e.g., parenterally, such as intravenously, to the subject a therapeutically effective amount of a highly bioavailable composition comprising CoQ10 as described herein.

### I. Definition

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. The term “such as” is used herein to mean, and is used interchangeably, with the phrase “such as but not limited to.”

The articles “a” and “an” are used herein to refer to one or to more than one (i.e. to at least one) of the grammatical object of the article. By way of example, “an element” means one element or more than one element.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. As used herein, “one or more” is understood as each value 1, 2, 3, 4, 5, 6, 7, 8, 9, 10, and any value greater than 10.

Generally, nomenclatures used in connection with, and techniques of, cell and tissue culture, molecular biology, immunology, microbiology, genetics and protein and nucleic acid chemistry described herein are those well-known and commonly used in the art. The methods and techniques of the present invention are generally performed according to conventional methods well known in the art and as described in various general and more specific references that are cited and discussed throughout the present specification unless otherwise indicated. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As used herein, the term “primary coenzyme Q10 (CoQ10) deficiency” refers to a group of conditions characterized by a reduction of CoQ10 levels in tissues or cultured cells associated with pathogenic variants (e.g., loss of function) in one of the ten genes involved in the biosynthesis of CoQ10, i.e., PDSS1, PDSS2, COQ2, COQ4, COQ5, COQ6, COQ7, COQ8A, COQ8B, and COQ9. Primary CoQ10 deficiency can affect any part of the body, but particularly the brain, muscle and kidney tissues, as a consequence of their high energy demands. The severity and time frame of symptoms are variable; severe symptoms may be evident in infancy, whereas mild symptoms may not become apparent until the individual is in their 60s. CoQ10 deficiency in brain tissue can cause ataxia, together with a range of other neurological manifestations. CoQ10 deficiency in kidney tissue results in nephrotic syndrome and renal dysfunction, and deficiency in cardiac tissue results in weakened heart muscle, characteristic of hypertrophic cardiomyopathy.

As used herein, the terms “treat,” “treating” or “treatment” refer to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) of the state of disease, delay in onset or slowing of condition, disorder or disease progression; amelioration or palliation of the disease state, diminishing rate of or time to progression, and remission (whether partial or total). “Treatment” of a disorder can also mean eliciting a clinically significant response without excessive levels of side effects, and/or prolonging survival as compared to expected survival in the absence of treatment. Treatment need not be curative.

A “therapeutically effective amount” means the amount of a compound that, when administered to a patient for treating a disease, is sufficient to effect such treatment for the disease, e.g., the amount of such a substance that produces some desired local or systemic effect at a reasonable benefit/risk ratio applicable to any treatment, e.g., is sufficient to ameliorate at least one sign or symptom of the disease, e.g., to increase the plasma level of CoQ10 in the subject. The “therapeutically effective amount” will vary depending on the compound, its therapeutic index, solubility, the disease and its severity and the age, weight, etc., of the patient to be treated, and the like. For example, certain compounds discovered by the methods of the present invention may be administered in a sufficient amount to produce a reasonable benefit/risk ratio applicable to such treatment. Administration of a therapeutically effective amount of a compound may require the administration of more than one dose of the compound.

As used herein, the terms “administer”, “administering” or “administration” include any method of delivery of a pharmaceutical composition or agent into a subject's system or to a particular region in or on a subject. In certain embodiments of the invention, the composition is administered by parenteral delivery, including, e.g., intravenous, intramuscular, subcutaneous, intramedullary injections, as well as intrathecal, direct intraventricular, intraperitoneal, intranasal, or intraocular injections. In certain embodiments, the composition is administered intravenously. In one embodiment, the compositions provided herein may be administered by injecting directly to a tissue. In some embodiments, the composition is administered by injection or infusion. In certain embodiments, administration is systemic. In certain embodiments, administration is local. Administering a composition can be performed by a number of people working in concert. Administering a composition includes, for example, prescribing a composition to be administered to a subject and/or providing instructions, directly or through another, to take a specific composition, either by self-delivery, e.g., subcutaneous delivery, intravenous delivery through a central line, etc; or for delivery by a trained professional, e.g., intravenous delivery, intramuscular delivery, etc.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, “opsonization” refers to the process by which a lipophilic bioactive agent as described herein is marked for ingestion and destruction by a phagocyte. Opsonization involves the binding of an opsonin to bioactive agent. After opsonin binds to the membrane, phagocytes are attracted to the active agent. An opsonin is any molecule that acts as a binding enhancer for the process of phagocytosis.

As used herein, the term “opsonization reducer” refers to any agent that works in conjunction with the active agent to reduce the ability of opsonins to act as a binding enhancer for the process of phagocytosis.

In accordance with the present disclosure, a formulation is provided for improved administration of lipophilic bioactive agents, which may also be referred to herein as hydrophobic bioactive agents. As used herein, a “lipophilic bioactive agent” or “hydrophobic bioactive agent” includes an agent that is insoluble or is substantially insoluble in water, e.g., Coenzyme CoQ10. Specifically, lipophilic bioactive agents, as used herein, will have a solubility in water that is less than about 1 part of bioactive drug in about 1000 parts of water.

As used herein, the term “colloidal” refers to a state of subdivision, implying that the molecules or polymolecular particles dispersed in a medium have at least in one direction a dimension roughly between 1-nm and 1-μm.

As used herein, a “dispersion” or “colloidal dispersion” refers to a system in which particles of colloidal size of any nature (e.g., solid, liquid or gas) are dispersed in a continuous phase of a different composition or state. In intravenous drug delivery the continuous phase is substantially water and the dispersed particles can be solid (a suspension) or an immiscible liquid (emulsion).

As used herein, a “super-cooled melt” refers to the state of the active agent after homogenization wherein at a temperature below the melting point of the bulk material of the active agent, the colloidal particles are not in a solid or crystalline form but rather in an amorphous state.

As used herein, a “lyoprotectant” refers to pharmaceutically acceptable excipients, which protect the dispersed active agent against destabilizing conditions during the lyophilisation process, subsequent storage and reconstitution.

The terms “colloidal particles,” “dispersion particles,” “nano-dispersion particles,” and “colloidal dispersion particles” are all used interchangeably herein and refer to the dispersed form of the active agent into nano-particles either in the bulk state or in a melted state.

The term “CoQ10 supplement”, as used herein, refers to an over-the-counter oral CoQ10 supplement. The CoQ10 supplement may come in a capsule or tablet form.

### II. Methods of Treatment

The present invention is based, at least in part, on the unexpected and surprising discovery that administration of a highly bioavailable CoQ10 composition results in a significant increase in the plasma level of CoQ10 in patients. Specifically, the present inventors have successfully demonstrated that an about 200-fold increase in the plasma concentration of CoQ10 was achieved upon administration (e.g., intravenous and/or oral administration) of the CoQ10 composition over normal plasma levels.

Accordingly, the present invention provides methods for treating primary CoQ10 deficiency in a subject in need thereof, by administering to the subject a therapeutically effective amount of a composition comprising CoQ10. In one embodiment, the composition is administered parenterally. In one embodiment, the composition is administered intravenously.

The present invention further provides methods for treating primary CoQ10 deficiency in a subject in need thereof, by (i) administering parenterally to the subject a therapeutically effective amount of a composition comprising CoQ10, and (ii) administering orally a therapeutically effective amount of the composition comprising CoQ10, thereby treating the subject.

Coenzyme Q10 (CoQ10) is a lipid involved in many cellular processes and necessary for mitochondrial electron transport, in particular. CoQ10 is composed of a redox-active benzoquinone head group and species-specific isoprenoid side chain (10 subunits in humans). CoQ10 takes three forms depending on the redox state of the benzoquinone ring; oxidized (Ubiquinone), fully-reduced (CoQ10H2, Ubiquinol), and semi-reduced (Ubiquinone+, Semiubiquinone) forms. Biosynthesis of CoQ10 in humans is complex and not completely deciphered, but it is known to involve PDSS1, PDSS2, COQ2, COQ3, COQ4, COQ5, COQ6, COQ7, COQ9, ADCK1, ADCK2, ADCK3, ADCK4, ADCK5, COQ10A, FDX1L and FDXR genes in human.

Primary CoQ10 deficiency is caused by mutations in one or more CoQ10 biosynthetic genes, which leads to CoQ10 deficiency and consequently manifests as disorders related to the different functions of CoQ10. The clinical manifestation of primary CoQ10 deficiency is heterogeneous, and is clustered in five main phenotypes: encephalomyopathy, cerebellar ataxia, a severe infantile multisystemic form, nephropathy, and isolated myopathy. The spectrum of clinical phenotypes observed in patients, however, is much wider and includes other clinical manifestations such as cardiomyopathy, optic atrophy, and deafness, and many different combinations of symptoms. Clinical symptoms due to primary CoQ10 deficiency also vary widely with respect to time of onset (from birth to old age), severity (from fatal multisystemic disorder to milder, tissue specific manifestations), affected tissue and clinical response to existing CoQ10 oral supplementation (see, e.g., Desbats et al., Genetic bases and clinical manifestations of coenzyme Q10 (CoQ10) deficiency, J Inherit Metab Dis (2015) 38:145-156, the entire contents of which are incorporated herein by reference).

In some embodiments of the methods provided herein, the subject is or has been determined as having primary CoQ10 deficiency. In some embodiments, the subject is suspected to have primary CoQ10 deficiency. In some embodiments, the subject has suffered and/or is suffering from clinical symptoms due to primary CoQ10 deficiency. In some embodiments, the subject is expected to develop clinical symptoms due to primary CoQ10 deficiency.

In some embodiments, the subject is or has been determined or confirmed to be CoQ10-deficient. In some embodiments, the CoQ10 deficiency is determined or confirmed by one or more methods comprising biochemical measurement of CoQ10 in muscle tissue and/or histologic examination of muscle biopsy. In some embodiments, the CoQ10 deficiency is determined or confirmed by one or methods comprising measurement of CoQ10 levels in plasma.

In some embodiments, the subject is or has been determined to have a loss of function, e.g., a loss of function mutation, in one or more genes that participate in the biosynthesis of CoQ10 (i.e., CoQ10 biosynthetic genes). In certain embodiments, the subject is or has been determined to have a loss of function, e.g., loss-of-function mutation, in one or more genes selected from the group consisting of PDSS1, PDSS2, COQ2, COQ4, COQ5, COQ6, COQ7, COQ8A, COQ8B, and COQ9.

In some embodiments, a subject is or has been determined as having primary CoQ10 deficiency based on the presence of a loss of function, e.g., loss-of-function mutation, in one or more CoQ10 biosynthetic genes. In certain embodiments, the subject is or has been determined as having primary CoQ10 deficiency based on the presence of a loss of function, e.g., a loss-of-function mutation, in one or more CoQ10 biosynthetic genes selected from the group consisting of PDSS1, PDSS2, COQ2, COQ4, COQ5, COQ6, COQ7, COQ8A, COQ8B, and COQ9.

In some embodiments, the subject has a loss of function, e.g., loss-of-function mutation, in PDSS1. In some embodiments, the subject has a loss of function, e.g., loss-of-function mutation, in PDSS2. In some embodiments, the subject has a loss of function, e.g., loss-of-function mutation, in COQ2. In some embodiments, the subject has a loss of function, e.g., loss-of-function mutation, in COQ4. In some embodiments, the subject has a loss of function, e.g., loss-of-function mutation, in COQ5. In some embodiments, the subject has a loss of function, e.g., loss-of-function mutation, in COQ6. In some embodiments, the subject has a loss of function, e.g., loss-of-function mutation, in COQ7. In some embodiments, the subject has a loss of function, e.g., loss-of-function mutation, in COQ8A. In some embodiments, the subject has a loss of function, e.g., loss-of-function mutation, in COQ8B. In some embodiments, the subject has a loss of function, e.g., loss-of-function mutation, in COQ9.

In some embodiments, the CoQ10 biosynthetic gene is PDSS1. PDSS1, also known as decaprenyl diphosphate synthase subunit 1, is involved in conversion of farnesyl diphosphate to decaprenal diphosphate. Human PDSS1 nucleotide and amino acid sequences can be found at GenBank Accession No. NM_014317.5, NM_NM_001321978.2, or NM_001321979.2, incorporated herein by reference.

In some embodiments, the CoQ10 biosynthetic gene is PDSS2. PDSS2, also known as decaprenyl diphosphate synthase subunit 2, is involved in conversion of farnesyl diphosphate to decaprenal diphosphate. Human PDSS2 nucleotide and amino acid sequences can be found at GenBank Accession No. NM_020381.4, incorporated herein by reference.

In some embodiments, the CoQ10 biosynthetic gene is COQ2. COQ2, also known as coenzyme Q2 or polyprenyltransferase, was the first gene identified to be associated with CoQ10 deficiency. Human COQ2 nucleotide and amino acid sequences can be found at GenBank Accession No. NM_015697.9 or NM_001358921.2, incorporated herein by reference.

In some embodiments, the CoQ10 biosynthetic gene is COQ4. COQ4, also known as coenzyme 4, is involved in organization of a complex of multiple biosynthetic enzymes. Human COQ4 nucleotide and amino acid sequences can be found at GenBank Accession No.

NM_016035.5 or NM_001305942.2, incorporated herein by reference.

In some embodiments, the CoQ10 biosynthetic gene is COQ5. COQ5, also known as coenzyme 5, is a C-methyl transferase. Human COQ5 nucleotide and amino acid sequences can be found at GenBank Accession No. NM_032314.4, incorporated herein by reference.

In some embodiments, the CoQ10 biosynthetic gene is COQ6. COQ6, also known as coenzyme 6, is a mono-oxigenase. Human COQ6 nucleotide and amino acid sequences can be found at GenBank Accession No. NM_182476.3, NM_182480.3, NM_001425255.1, NM_001425256.1, NM_001425258.1, NM_001425259.1, NM_001425261.1 or NM_001425262.1, incorporated herein by reference.

In some embodiments, the CoQ10 biosynthetic gene is COQ7. COQ7, also known as coenzyme 7, is a hydroxylase. Human COQ7 nucleotide and amino acid sequences can be found at GenBank Accession No. NM_016138.5, NM_001190983.2, NM_001370489.1, NM_001370490.1, NM_001370491.1, NM_001370492.1, NM_001370493.1, NM_001370494.1, or NM_001370495.1, incorporated herein by reference.

In some embodiments, the CoQ10 biosynthetic gene is COQ8A, also known as coenzyme Q8A or ADCK3. Human COQ8A nucleotide and amino acid sequences can be found at GenBank Accession No. NM_020247.5, incorporated herein by reference.

In some embodiments, the CoQ10 biosynthetic gene is COQ8B, also known as coenzyme Q8B or ADCK4. Human COQ8B nucleotide and amino acid sequences can be found at GenBank Accession No. NM_024876.4 or NM_001142555.3, incorporated herein by reference.

In some embodiments, the CoQ10 biosynthetic gene is COQ9, also known as coenzyme Q9. Human COQ9 nucleotide and amino acid sequences can be found at GenBank Accession No. NM_020312.4, incorporated herein by reference.

Any appropriate methods or techniques can be used to determine whether or not a subject has a loss of function in one or more genes involved in a homologous recombination repair pathway. As used herein, a “loss of function” in a gene refers to a change or modification or a difference in the sequence of at least one copy of either or both of a gene relative to an appropriate reference sequence (e.g., a wild type reference and/or a sequence that is present in healthy cells in the subject), such as, the presence, absence, or extent/level of some physical, chemical, or genetic characteristic of a gene or its expression product(s). Such characteristics include, but are not limited to, mutations (e.g., single nucleotide variants, small insertions and deletions (indels), or large deletions and duplications), copy number variants (CNV) (e.g., copy number loss, or copy number gain), and methylation (e.g., promoter methylation) in the gene.

These may be assayed directly (e.g., by sequencing a gene, or by assaying a gene's expression level) or determined indirectly (e.g., assaying the level of a gene or genes whose expression level is correlated to the expression level of the gene of interest). Those skilled in the art are familiar with various techniques for determining the status of a gene or protein including, but not limited to, whole genome or gene-specific sequencing, locus-specific genotyping (e.g., SNP arrays), large-rearrangement analysis, CNV analysis, microarray mRNA expression analysis, quantitative real-time PCR (qRT-PCR, e.g., TaqMan), immunoanalysis (e.g., ELISA, immunohistochemistry), etc. The methods of the invention may be practiced independent of the particular technique used. In some embodiments, multiple techniques are used to confirm the status of the genes.

In some embodiments, a subject (e.g., a human) can be identified as having a loss of function in one or more CoQ10 biosynthetic genes using standard diagnostic techniques. Genetic tests are available, and known by those of skill in the art. Single nucleotide variants and small insertions and deletions (indels) may be identified by polymerase chain reaction (PCR) and nucleotide sequencing. Large deletions and duplications in the genes may be detected using multiplex PCR. In some embodiments, gene mutations can be detected using next generation sequencing methods, e.g., the Foundation One Liquid CDx test approved for detecting gene alterations.

In some embodiments, the subject has suffered clinical symptoms due to primary CoQ10 deficiency. In some other embodiments, the subject is anticipated to have or develop clinical symptoms due to primary CoQ10 deficiency.

In some embodiments, administration, e.g., parenteral (e.g., IV) and/or oral administration of the CoQ10 composition as described herein results in a CoQ10 plasma concentration of about 10 μg/mL to about 3000 μg/mL, about 10 μg/mL to about 2500 μg/mL, about 10 μg/mL to about 2000 μg/mL, about 10 μg/mL to about 1000 μg/mL, about 10 μg/mL to about 500 μg/mL, about 10 μg/mL to about 400 μg/mL, about 10 μg/mL to about 300 μg/mL, about 10 μg/mL to about 200 μg/mL, about 10 μg/mL to about 100 μg/mL, about 20 μg/mL to about 2500 μg/mL, about 20 μg/mL to about 2000 μg/mL, about 30 μg/mL to about 2500 μg/mL, about 30 μg/mL to about 2000 μg/mL, about 40 μg/mL to about 2500 μg/mL, about 40 μg/mL to about 2000 μg/mL, about 50 μg/mL to about 2500 μg/mL, about 50 μg/mL to about 2000 μg/mL, about 60 μg/mL to about 2500 μg/mL, about 60 μg/mL to about 2000 μg/mL, about 70 μg/mL to about 2500 μg/mL, about 70 μg/mL to about 2000 μg/mL, about 80 μg/mL to about 2500 μg/mL, about 80 μg/mL to about 2000 μg/mL, about 90 μg/mL to about 2500 μg/mL, about 90 μg/mL to about 2000 μg/mL, about 100 μg/mL to about 2500 μg/mL, about 100 μg/mL to about 2000 μg/mL, about 100 μg/mL to about 1500 μg/mL, about 100 μg/mL to about 1000 μg/mL, about 100 μg/mL to about 500 μg/mL, about 500 μg/mL to about 1000 μg/mL, about 1000 μg/mL to about 1500 μg/mL, about 1500 μg/mL to about 2000 μg/mL, about 2000 μg/mL to about 2500 μg/mL, or about 2500 μg/mL to about 3000 μg/mL, in the subject.

In some embodiments, administration, e.g., parenteral (e.g., IV) and/or oral administration of the CoQ10 composition as described herein results in a CoQ10 plasma concentration of at least about 10 μg/mL, e.g., at least about 15 μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, about 100 μg/mL, about 125 μg/mL, about 150 μg/mL, about 175 μg/mL, about 200 μg/mL, about 300 μg/mL, about 400 μg/mL, about 500 μg/mL, about 600 μg/mL, about 700 μg/mL, about 800 μg/mL, about 900 μg/mL, about 1000 μg/mL, about 1500 μg/mL, about 2000 μg/mL, about 2500 μg/mL, or about 3000 μg/mL in the subject.

In some embodiments, administration, e.g., parenteral (e.g., IV) and/or oral administration of the CoQ10 composition as described herein results in a CoQ10 plasma concentration of greater than about 10 μg/mL, e.g., greater than about 15 μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, about 100 μg/mL, about 125 μg/mL, about 150 μg/mL, about 175 μg/mL, about 200 μg/mL, about 300 μg/mL, about 400 μg/mL, about 500 μg/mL, about 600 μg/mL, about 700 μg/mL, about 800 μg/mL, about 900 μg/mL, about 1000 μg/mL, about 1500 μg/mL, about 2000 μg/mL, about 2500 μg/mL, or about 3000 μg/mL in the subject.

In some embodiments, a plasma concentration of at least about 10 μg/mL for CoQ10 is achieved in the subject. In some embodiments, a plasma concentration of at least about 15 μg/mL for CoQ10 is achieved in the subject. In some embodiments, a plasma concentration of at least about 20 μg/mL for CoQ10 is achieved in the subject. In some embodiments, a plasma concentration of at least about 25 μg/mL for CoQ10 is achieved in the subject. In some embodiments, a plasma concentration of at least about 30 μg/mL for CoQ10 is achieved in the subject. In some embodiments, a plasma concentration of at least about 35 μg/mL for CoQ10 is achieved in the subject. In some embodiments, a plasma concentration of at least about 40 μg/mL for CoQ10 is achieved in the subject. In some embodiments, a plasma concentration of at least about 45 μg/mL for CoQ10 is achieved in the subject. In some embodiments, a plasma concentration of at least about 50 μg/mL for CoQ10 is achieved in the subject. In some embodiments, a plasma concentration of at least about 55 μg/mL for CoQ10 is achieved in the subject. In some embodiments, a plasma concentration of at least about 60 μg/mL for CoQ10 is achieved in the subject. In some embodiments, a plasma concentration of at least about 65 μg/mL for CoQ10 is achieved in the subject. In some embodiments, a plasma concentration of at least about 70 μg/mL for CoQ10 is achieved in the subject. In some embodiments, a plasma concentration of at least about 75 μg/mL for CoQ10 is achieved in the subject. In some embodiments, a plasma concentration of at least about 80 μg/mL for CoQ10 is achieved in the subject. In some embodiments, a plasma concentration of at least about 85 g/mL for CoQ10 is achieved in the subject. In some embodiments, a plasma concentration of at least about 90 μg/mL for CoQ10 is achieved in the subject. In some embodiments, a plasma concentration of at least about 95 μg/mL for CoQ10 is achieved in the subject. In some embodiments, a plasma concentration of at least about 100 μg/mL for CoQ10 is achieved in the subject. In some embodiments, a plasma concentration of at least about 125 μg/mL for CoQ10 is achieved in the subject. In some embodiments, a plasma concentration of at least about 150 μg/mL for CoQ10 is achieved in the subject. In some embodiments, a plasma concentration of at least about 175 μg/mL for CoQ10 is achieved in the subject. In some embodiments, a plasma concentration of at least about 200 μg/mL for CoQ10 is achieved in the subject.

In some embodiments, a plasma concentration of about 0.01 mM to 3.0 mM, about 0.01 mM to 2.5 mM, about 0.01 mM to 2.0 mM, about 0.01 mM to 1.0 mM, about 0.01 mM to 0.5 mM, about 0.01 mM to 0.4 mM, about 0.01 mM to 0.3 mM, about 0.01 mM to 0.2 mM, about 0.01 mM to 0.1 mM, about 0.02 mM to 2.0 mM, about 0.03 mM to 2.0 mM, about 0.04 mM to 2.0 mM, about 0.05 mM to 2.0 mM, about 0.06 mM to 2.0 mM, about 0.07 mM to 2.0 mM, about 0.08 mM to 2.0 mM, about 0.09 mM to 2.0 mM, about 0.1 mM to 2.0 mM, about 0.1 mM to 1.9 mM, about 0.1 mM to 1.8 mM, about 0.1 mM to 1.7 mM, about 0.1 mM to 1.6 mM, about 0.1 mM to 1.5 mM, about 0.1 mM to 1.4 mM, about 0.1 mM to 1.3 mM, about 0.1 mM to 1.2 mM, about 0.1 mM to 1.1 mM, about 0.1 mM to 1.0 mM, about 0.15 mM to 1.0 mM, about 0.2 mM to 1.0 mM, about 0.25 mM to 1.0 mM, about 0.3 mM to 1.0 mM, about 0.35 mM to 1.0 mM, about 0.4 mM to 1.0 mM, about 0.45 mM to 1.0 mM, about 0.5 mM to 1.0 mM, about 0.55 mM to 1.0 mM, about 0.6 mM to 1.0 mM, about 0.65 mM to 1.0 mM, about 0.7 mM to 1.0 mM, about 0.75 mM to 1.0 mM, about 0.8 mM to 1.0 mM, about 0.85 mM to 1.0 mM, or about 0.9 mM to 1.0 mM, for CoQ10 is achieved in the subject.

In some embodiments, the composition comprising CoQ10 is administered (e.g., parenterally, e.g., intravenously, and/or orally) in a dose and frequency such that a plasma concentration of CoQ10 of at least about 10 μM, at least about 20 μM, at least about 30 μM, at least about 40 μM, at least about 50 μM, at least about 60 μM, at least about 70 μM, at least about 80 μM, at least about 90 μM, at least about 100 μM, at least about 150 μM, at least about 200 μM, at least about 300 μM, at least about 400 μM, at least about 500 M, at least about 600 μM, at least about 700 μM, at least about 800 μM, at least about 900 μM, at least about 1 mM, or at least about 1.5 mM, is achieved in the subject. In some embodiments, the composition comprising CoQ10 is administered (e.g., parenterally, e.g., intravenously, and/or orally) in a dose and frequency such that a plasma concentration of CoQ10 of about 10 μM to 1.5 mM or about 10 μM to 1 mM is achieved in the subject. In some embodiments, the composition comprising CoQ10 is administered in a dose and frequency such that a plasma concentration of CoQ10 of between about 10 μM to 1 mM, about 50 μM to 1 mM, about 100 μM to 1 mM, about 200 μM to 1 mM, about 300 M to 1 mM, about 400 μM to 1 mM, about 500 μM to 1 mM, about 600 μM to 1 mM, about 700 μM to 1 mM, about 800 μM to 1 mM, or about 900 μM to 1 mM is achieved in the subject. In some embodiments, the composition comprising CoQ10 is administered (e.g., parenterally, e.g., intravenously, and/or orally) in a dose and frequency such that a plasma concentration of CoQ10 of between about 10 μM to 900 μM, about 10 μM to 800 μM, about 10 μM to 700 μM, about 10 μM to 600 μM, about 10 μM to 500 μM, about 10 μM to 400 μM, or about 10 μM to 300 μM, about 10 μM to 200 μM, about 10 μM to 100 μM, about 50 μM to 200 μM, about 50 μM to 800 μM, about 50 μM to 700 μM, about 50 μM to 600 μM, about 50 μM to 500 μM, about 50 μM to 400 μM, or about 50 μM to 300 μM is achieved in the subject.

In some embodiments, administration, e.g., parenteral (e.g., IV) and/or oral administration of the composition comprising CoQ10 results in a plasma concentration of CoQ10 that is at least 10-fold greater than the plasma concentration of CoQ10 of a normal healthy individual. In some embodiments, administration, e.g., parenteral (e.g., IV) and/or oral administration of the composition comprising CoQ10 results in a plasma concentration of CoQ10 that is at least 20-fold greater than the plasma concentration of CoQ10 of a normal healthy individual. In some embodiments, administration, e.g., parenteral (e.g., IV) and/or oral administration of the composition comprising CoQ10 results in a plasma concentration of CoQ10 that is at least 30-fold greater than the plasma concentration of CoQ10 of a normal healthy individual. In some embodiments, administration, e.g., parenteral (e.g., IV) and/or oral administration of the composition comprising CoQ10 results in a plasma concentration of CoQ10 that is at least 40-fold greater than the plasma concentration of CoQ10 of a normal healthy individual. In some embodiments, administration, e.g., parenteral (e.g., IV) and/or oral administration of the composition comprising CoQ10 results in a plasma concentration of CoQ10 that is at least 50-fold greater than the plasma concentration of CoQ10 of a normal healthy individual. In some embodiments, administration, e.g., parenteral (e.g., IV) and/or oral administration of the composition comprising CoQ10 results in a plasma concentration of CoQ10 that is at least 75-fold greater than the plasma concentration of CoQ10 of a normal healthy individual. In some embodiments, administration, e.g., parenteral (e.g., IV) and/or oral administration of the composition comprising CoQ10 results in a plasma concentration of CoQ10 that is at least 100-fold greater than the plasma concentration of CoQ10 of a normal healthy individual. In some embodiments, administration, e.g., parenteral (e.g., IV) and/or oral administration of the composition comprising CoQ10 results in a plasma concentration of CoQ10 that is at least 150-fold greater than the plasma concentration of CoQ10 of a normal healthy individual. In some embodiments, administration, e.g., parenteral (e.g., IV) and/or oral administration of the composition comprising CoQ10 results in a plasma concentration of CoQ10 that is at least 200-fold greater than the plasma concentration of CoQ10 of a normal healthy individual. In some embodiments, administration, e.g., parenteral (e.g., IV) and/or oral administration of the composition comprising CoQ10 results in a plasma concentration of CoQ10 that is at least 500-fold greater than the plasma concentration of CoQ10 of a normal healthy individual. In some embodiments, administration, e.g., parenteral (e.g., IV) and/or oral administration of the composition comprising CoQ10 results in a plasma concentration of CoQ10 that is at least 1000-fold greater than the plasma concentration of CoQ10 of a normal healthy individual. In some embodiments, administration, e.g., parenteral (e.g., IV) and/or oral administration of the composition comprising CoQ10 results in a plasma concentration of CoQ10 that is at least 2000-fold greater than the plasma concentration of CoQ10 of a normal healthy individual. The CoQ10 composition, as described herein, can be administered to a subject in any appropriate amount, at any appropriate frequency, and for any appropriate duration effective to achieve a desired outcome, such as the target plasma concentrations of CoQ10 described herein.

In some embodiments, administration, e.g., parenteral (e.g., IV) and/or oral administration of the CoQ10 composition as described herein increases the concentration of CoQ10 in the brain, heart, kidney and/or muscle tissues of the subject.

In some embodiments, the methods restore the level of CoQ10 in the subject having primary CoQ10 deficiency to a normal physiological level or above the physiological level in the brain, heart, kidney and/or muscle tissues of the subject.

In some embodiments, the methods result in a change in the levels of lactate, succinate and/or citrate in the brain, heart, kidney and/or muscle tissues of the subject.

In some embodiments, administration, e.g., parenteral (e.g., IV) and/or oral administration of the CoQ10 composition as described herein results in a CoQ10 concentration of about 0.1 μg/mg to about 100 μg/mg in the brain, heart, kidney, and/or muscle tissues of the subject. In some embodiments, administration, e.g., parenteral (e.g., IV) and/or oral administration of the CoQ10 composition as described herein results in a CoQ10 concentration of about 0.1 μg/mg to about 50 μg/mg in the brain, heart, kidney, and/or muscle tissues of the subject. In some embodiments, administration, e.g., parenteral (e.g., IV) and/or oral administration of the CoQ10 composition as described herein results in a CoQ10 concentration of about 50 μg/mg to about 100 μg/mg in the brain, heart, kidney, and/or muscle tissues of the subject. In some embodiments, administration, e.g., parenteral (e.g., IV) and/or oral administration of the CoQ10 composition as described herein results in a CoQ10 concentration of about 0.1 μg/mg to about 25 μg/mg in the brain, heart, kidney, and/or muscle tissues of the subject. In some embodiments, administration, e.g., parenteral (e.g., IV) and/or oral administration of the CoQ10 composition as described herein results in a CoQ10 concentration of about 25 μg/mg to about 50 μg/mg in the brain, heart, kidney, and/or muscle tissues of the subject. In some embodiments, administration, e.g., parenteral (e.g., IV) and/or oral administration of the CoQ10 composition as described herein results in a CoQ10 concentration of about 50 μg/mg to about 75 μg/mg in the brain, heart, kidney, and/or muscle tissues of the subject. In some embodiments, administration, e.g., parenteral (e.g., IV) and/or oral administration of the CoQ10 composition as described herein results in a CoQ10 concentration of about 75 μg/mg to about 100 μg/mg in the brain, heart, kidney, and/or muscle tissues of the subject.

In some embodiments, administration, e.g., parenteral (e.g., IV) and/or oral administration of the CoQ10 composition as described herein results in a CoQ10 concentration of at least about 0.1 μg/mg, e.g., about 0.5 μg/mg, about 1 μg/mg, about 5 μg/mg, about 10 μg/mg, about 15 μg/mg, about 20 μg/mg, about 25 μg/mg, about 30 μg/mg, about 35 μg/mg, about 40 μg/mg, about 45 μg/mg, about 50 μg/mg, about 55 μg/mg, about 60 μg/mg, about 65 μg/mg, about 70 μg/mg, about 75 μg/mg, about 80 μg/mg, about 85 μg/mg, about 90 μg/mg, about 95 μg/mg, about 100 μg/mg, about 125 μg/mg, about 150 μg/mg, about 175 μg/mg, or about 200 μg/mg in the brain, heart, kidney, and/or muscle tissues of the subject.

In some embodiments, administration, e.g., parenteral (e.g., IV) and/or oral administration of the CoQ10 composition as described herein results in a CoQ10 concentration of greater than about 0.1 μg/mg, e.g., about 0.5 μg/mg, about 1 μg/mg, about 5 μg/mg, about 10 μg/mg, about 15 μg/mg, about 20 μg/mg, about 25 μg/mg, about 30 μg/mg, about 35 μg/mg, about 40 μg/mg, about 45 μg/mg, about 50 μg/mg, about 55 μg/mg, about 60 μg/mg, about 65 μg/mg, about 70 μg/mg, about 75 μg/mg, about 80 μg/mg, about 85 μg/mg, about 90 μg/mg, about 95 μg/mg, about 100 μg/mg, about 125 μg/mg, about 150 μg/mg, about 175 μg/mg, or about 200 μg/mg in the brain, heart, kidney, and/or muscle tissues of the subject. In some embodiments, a concentration of at least about 0.1 μg/mg for CoQ10 is achieved in the brain, heart, kidney, and/or muscle tissues of the subject. In some embodiments, a concentration of at least about 0.5 μg/mg for CoQ10 is achieved in the brain, heart, kidney, and/or muscle tissues of the subject. In some embodiments, a concentration of at least about 1 μg/mg for CoQ10 is achieved in the brain, heart, kidney, and/or muscle tissues of the subject. In some embodiments, a concentration of at least about 5 μg/mg for CoQ10 is achieved in the brain, heart, kidney, and/or muscle tissues of the subject. In some embodiments, a concentration of at least about 10 μg/mg for CoQ10 is achieved in the brain, heart, kidney, and/or muscle tissues of the subject. In some embodiments, a concentration of at least about 15 μg/mg for CoQ10 is achieved in the brain, heart, kidney, and/or muscle tissues of the subject. In some embodiments, a concentration of at least about 20 μg/mg for CoQ10 is achieved in the brain, heart, kidney, and/or muscle tissues of the subject. In some embodiments, a concentration of at least about 25 μg/mg for CoQ10 is achieved in the brain, heart, kidney, and/or muscle tissues of the subject. In some embodiments, a concentration of at least about 30 μg/mg for CoQ10 is achieved in the brain, heart, kidney, and/or muscle tissues of the subject. In some embodiments, a concentration of at least about 35 μg/mg for CoQ10 is achieved in the brain, heart, kidney, and/or muscle tissues of the subject. In some embodiments, a concentration of at least about 40 μg/mg for CoQ10 is achieved in the brain, heart, kidney, and/or muscle tissues of the subject. In some embodiments, a concentration of at least about 45 μg/mg for CoQ10 is achieved in the brain, heart, kidney, and/or muscle tissues of the subject. In some embodiments, a concentration of at least about 50 μg/mg for CoQ10 is achieved in the brain, heart, kidney, and/or muscle tissues of the subject. In some embodiments, a concentration of at least about 55 μg/mg for CoQ10 is achieved in the brain, heart, kidney, and/or muscle tissues of the subject. In some embodiments, a concentration of at least about 60 μg/mg for CoQ10 is achieved in the brain, heart, kidney, and/or muscle tissues of the subject. In some embodiments, a concentration of at least about 65 μg/mg for CoQ10 is achieved in the brain, heart, kidney, and/or muscle tissues of the subject. In some embodiments, a concentration of at least about 70 μg/mg for CoQ10 is achieved in the brain, heart, kidney, and/or muscle tissues of the subject. In some embodiments, a concentration of at least about 75 μg/mg for CoQ10 is achieved in the brain, heart, kidney, and/or muscle tissues of the subject. In some embodiments, a concentration of at least about 80 μg/mg for CoQ10 is achieved in the brain, heart, kidney, and/or muscle tissues of the subject. In some embodiments, a concentration of at least about 85 μg/mg for CoQ10 is achieved in the brain, heart, kidney, and/or muscle tissues of the subject. In some embodiments, a concentration of at least about 90 μg/mg for CoQ10 is achieved in the brain, heart, kidney, and/or muscle tissues of the subject. In some embodiments, a concentration of at least about 95 μg/mg for CoQ10 is achieved in the brain, heart, kidney, and/or muscle tissues of the subject. In some embodiments, a concentration of at least about 100 μg/mg for CoQ10 is achieved in the brain, heart, kidney, and/or muscle tissues of the subject. In some embodiments, a concentration of at least about 125 μg/mg for CoQ10 is achieved in the brain, heart, kidney, and/or muscle tissues of the subject. In some embodiments, a concentration of at least about 150 μg/mg for CoQ10 is achieved in the brain, heart, kidney, and/or muscle tissues of the subject. In some embodiments, a concentration of at least about 175 μg/mg for CoQ10 is achieved in the brain, heart, kidney, and/or muscle tissues of the subject. In some embodiments, a concentration of at least about 200 μg/mg for CoQ10 is achieved in the brain, heart, kidney, and/or muscle tissues of the subject.

In some embodiments, administration, e.g., parenteral (e.g., IV) and/or oral administration of the composition comprising CoQ10 results in a concentration of CoQ10 in the brain, heart, kidney, and/or muscle tissues that is at least 1.1-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 1.2-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 1.3-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 1.4-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 1.5-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 1.6-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 1.7-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 1.8-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 1.9-fold greater than the concentration of CoQ10 in the brain, heart, kidney, and/or muscle tissues prior to administration of the CoQ10 composition as described herein.

In some embodiments, administration, e.g., parenteral (e.g., IV) and/or oral administration of the composition comprising CoQ10 results in a concentration of CoQ10 in the brain, heart, kidney, and/or muscle tissues that is at least 2-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 3-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 4-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 5-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 6-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 7-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 8-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 9-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 10-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 20-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 30-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 40-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 50-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 75-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 100-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 150-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 200-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 500-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 1000-fold greater than the concentration of CoQ10 in the brain, heart, kidney, and/or muscle tissues prior to administration of the CoQ10 composition as described herein. In some embodiments, administration, e.g., parenteral (e.g., IV) and/or oral administration of the composition comprising CoQ10 results in a concentration of CoQ10 in the brain, heart, kidney, and/or muscle tissues that is at least 2000-fold greater than the concentration of CoQ10 in the brain, heart, kidney, and/or muscle tissues prior to administration of the CoQ10 composition as described herein. The CoQ10 composition, as described herein, can be administered to a subject in any appropriate amount, at any appropriate frequency, and for any appropriate duration effective to achieve a desired outcome, such as the target concentrations of CoQ10 in the brain, heart, kidney, and/or muscle tissues described herein.

In some embodiments, administration, e.g., parenteral (e.g., IV) and/or oral administration, of the CoQ10 composition as described herein improves the clinical outcomes of the subject, e.g., the Scale for the Assessment and Rating of Ataxia (SARA) score, and the 9-Hole Peg Test (9HPT) in the subject.

SARA is a clinical scale which assesses a range of different impairments in cerebellar ataxia. The scale is made up of 8 items related to gait, stance, sitting, speech, finger-chase test, nose-finger test, fast alternating movements and heel-shin test with accumulative score ranging from 0 (no ataxia) to 40 (most severe ataxia). A minimal important change in SARA score is ≥1.

In some embodiments, administration, e.g., parenteral (e.g., IV) and/or oral administration, of the CoQ10 composition as described herein results in a decrease in a SARA score (e.g., a decrease of at least 1, 1.1, 1.2, 1.3, 1.4, 1.5, 1.6, 1.7, 1.8, 1.9, 2, 2.5, or 3), as compared to a baseline value, i.e., the SARA score measured in the subject prior to administration of the CoQ10 composition as described herein.

The 9HPT is used to measure finger dexterity in patients with various neurological diagnoses. In this test, a subject is asked to take pegs from a container, one by one, and place them into the nine holes on the board as quickly as possible. The subject must then remove the pegs from the holes, one by one, and replace them back into the container. Scores are based on the time taken to complete the test activity, recorded in seconds.

In some embodiments, administration, e.g., parenteral (e.g., IV) and/or oral administration, of the CoQ10 composition as described herein results in a decrease in a 9HPT score expressed as time to complete the test activity (e.g., at least an about 5%, 10%, 25%, or 50% decrease in a 9HPT score expressed as time to complete the test activity), as compared to a baseline value, i.e., the 9HPT score measured in the subject prior to administration of the CoQ10 composition as described herein.

In some embodiments, administration, e.g., parenteral (e.g., IV) and/or oral administration, of the CoQ10 composition as described herein results in a decrease in the time required to complete the 9HPT test activity (e.g., a decrease of at least 1, 2, 3, 4, 5, 6, 7, 8, 9, or 10 seconds), as compared to a baseline value, i.e., the time required to complete the test activity prior to administration of the CoQ10 composition as described herein.

In some embodiments, administration, e.g., parenteral (e.g., IV) and/or oral administration, of the CoQ10 composition as described herein also improves the patient-reported outcomes, e.g., Friedreich's Ataxia Rating Scale-Activities of Daily Living (FARS-ADL) and Patient Global Impression of Change (PGIC), and/or the observer-reported outcomes. FARS-ADL and PGIC are both used to measure the severity of ataxia and how it affects a patient's daily life. FARS-ADL is a scale that assesses a patient's ability to perform daily tasks, while PGIC is a measure of how a patient perceives their own change in condition.

In some embodiments, administration, e.g., parenteral (e.g., IV) and/or oral administration, of the CoQ10 composition as described herein results in a decrease in a FARS-ADL score (e.g., a decrease of at least 1, 1.5, 2, 2.5, 3, 3.5, 4, 4.5, 5, 5.5, or 6), as compared to a baseline value, i.e., the FARS-ADL score measured in the subject prior to administration of the CoQ10 composition as described herein.

In some embodiments, administration, e.g., parenteral (e.g., IV) and/or oral administration, of the CoQ10 composition as described herein results in an increase in a PGIC score (e.g., an increase of at least 1, 2, or 3), as compared to a baseline value, i.e., the PGIC score measured in the subject prior to administration of the CoQ10 composition as described herein.

In some embodiments, administration, e.g., parenteral (e.g., IV) and/or oral administration, of the CoQ10 composition as described herein also improves the gait and balance, dexterity, speech and cognition, and/or goal attainment, in the subject.

One skilled in the art would be able, by routine experimentation with the guidance provided herein, to determine what an effective, non-toxic amount of a CoQ10 composition would be for the purpose of treating primary CoQ10 deficiency. For example, a therapeutically active amount of a CoQ10 composition may vary according to factors such as the disease stage, age, sex, medical complications, and weight of the subject, and the ability of the CoQ10 composition to elicit a desired response in the subject. In certain embodiments, the subject is a human subject. The dosage regimen may be adjusted to provide the optimum therapeutic response.

In some embodiments, the CoQ10 composition is administered by intravenous infusion. In some embodiments, the CoQ10 composition is administered by intravenous infusion over about 1 hour, 2 hours, 3 hours, 4 hours or longer. In one embodiment, the CoQ10 composition is administered by infusion over about 4 hours.

In some embodiments, the CoQ10 composition is administered as a weight-based total weekly dose of CoQ10. In some embodiments, the CoQ10 composition is administered at a dose of about 5 mg/kg/week to about 1,000 mg/kg/week. In one embodiment, the CoQ10 composition is administered in the range of about 5 mg/kg/week to about 700 mg/kg/week. In one embodiment, the CoQ10 composition is administered in the range of about 5 mg/kg/week to about 400 mg/kg/week. In one embodiment, the CoQ10 composition is administered in the range of about 5 mg/kg/week to about 350 mg/kg/week. In one embodiment, the CoQ10 composition is administered in the range of about 5 mg/kg/week to about 200 mg/kg/week. In one embodiment, the CoQ10 composition is administered in the range of about 5 mg/kg/week to about 150 mg/kg/week. In one embodiment, the CoQ10 composition is administered in the range of about 5 mg/kg/week to about 100 mg/kg/week. In one embodiment, the CoQ10 composition is administered in the range of about 5 mg/kg/week to about 50 mg/kg/week. In one embodiment, the CoQ10 composition is administered in the range of about 10 mg/kg/week to about 50 mg/kg/week. In one embodiment, the CoQ10 composition is administered in the range of about 20 mg/kg/week to about 50 mg/kg/week.

In various embodiments, the CoQ10 composition is administered at a weekly dose of about 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg/kg, 320 mg/kg, 330 mg/kg, 340 mg/kg, 350 mg/kg, 360 mg/kg, 370 mg/kg, 380 mg/kg, 390 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1000 mg/kg. In various embodiments, the CoQ10 composition is administered at a weekly dose of about 50 mg/kg. In various embodiments, the CoQ10 composition is administered at a weekly dose of about 50 mg/kg.

In some embodiments, the CoQ10 composition is administered at a weekly dose of about 10 mg/kg to about 100 mg/kg. In some embodiments, the CoQ10 composition is administered at a weekly dose of about 25 mg/kg to about 75 mg/kg. In some embodiments, the CoQ10 composition is administered at a weekly dose of about 40 mg/kg to about 60 mg/kg.

In some embodiments, the CoQ10 composition is administered as a weight-based total daily dose of CoQ10. In some embodiments, the CoQ10 composition is administered at a dose of about 5 mg/kg/day to about 500 mg/kg/day. In one embodiment, the CoQ10 composition is administered in the range of about 5 mg/kg/day to about 400 mg/kg/day. In one embodiment, the CoQ10 composition is administered in the range of about 5 mg/kg/day to about 300 mg/kg/day. In one embodiment, the CoQ10 composition is administered in the range of about 5 mg/kg/day to about 200 mg/kg/day. In one embodiment, the CoQ10 composition is administered in the range of about 5 mg/kg/day to about 100 mg/kg/day. In one embodiment, the CoQ10 composition is administered in the range of about 5 mg/kg/day to about 50 mg/kg/day. In one embodiment, the CoQ10 composition is administered in the range of about 5 mg/kg/day to about 25 mg/kg/day.

In various embodiments, the CoQ10 composition is administered at a daily dose of about 2 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, or 300 mg/kg.

In one embodiment, the administered dose is at least about 1 mg/kg, at least about 5 mg/kg, at least about 10 mg/kg, at least about 12.5 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 75 mg/kg, at least about 100 mg/kg, at least about 125 mg/kg, at least about 150 mg/kg, at least about 175 mg/kg, at least about 200 mg/kg, at least about 300 mg/kg, or at least about 400 mg/kg of CoQ10.

It should be understood that ranges having any one of the foregoing values as the upper or lower limits are also intended to be part of this invention, e.g., about 50 mg/kg/day to about 200 mg/kg/day, or about 10 mg/kg/day to about 150 mg/kg/day.

In some embodiments, the CoQ10 composition is administered daily. In some embodiments, the CoQ10 composition is administered every other day. In some embodiments, the CoQ10 composition is administered once per day. In some embodiments, the CoQ10 composition is administered twice per day. In some embodiments, the CoQ10 composition is administered three times per day.

In some embodiments, the CoQ10 composition is administered once per week. In some embodiments, the CoQ10 composition is administered twice per week. In some embodiments, the CoQ10 composition is administered three times per week. In some embodiments, the CoQ10 composition is administered four times per week. In some embodiments, the CoQ10 composition is administered five times per week. In some embodiments, the CoQ10 composition is administered six times per week. In some embodiments, the CoQ10 composition is administered every two weeks. In some embodiments, the CoQ10 composition is administered every three weeks. In some embodiments, the CoQ10 composition is administered every four weeks.

In some embodiments, the CoQ10 composition is administered every month. In some embodiments, the CoQ10 composition is administered every two months. In some embodiments, the CoQ10 composition is administered every three months. In some embodiments, the CoQ10 composition is administered every four months. In some embodiments, the CoQ10 composition is administered every five months. In some embodiments, the CoQ10 composition is administered every six months.

In some embodiments, the CoQ10 composition is administered parenterally. In some embodiments, the CoQ10 composition is administered intravenously. In some embodiments, the CoQ10 composition is administered via infusion. In some embodiments, the CoQ10 composition is administered subcutaneously.

In some embodiments, the CoQ10 composition is administered parenterally weekly. In some embodiments, the CoQ10 composition is administered intravenously weekly. In some embodiments, the CoQ10 composition is administered via infusion weekly. In some embodiments, the CoQ10 composition is administered subcutaneously weekly.

In some embodiments, the CoQ10 composition is administered parenterally weekly at a dose of about 5 mg/kg to about 350 mg/kg. In some embodiments, the CoQ10 composition is administered intravenously weekly at a dose of about 5 mg/kg to about 350 mg/kg. In some embodiments, the CoQ10 composition is administered via infusion weekly at a dose of about 5 mg/kg to about 350 mg/kg. In some embodiments, the CoQ10 composition is administered subcutaneously weekly at a dose of about 5 mg/kg to about 350 mg/kg.

In some embodiments, the CoQ10 composition is administered parenterally (e.g., intravenously) in combination with administration of the CoQ10 composition orally. In some embodiments, the CoQ10 composition is administered orally according to an oral dosing schedule in combination with administration parenterally according to a parenteral dosing schedule. In some embodiments, the CoQ10 composition is administered orally weekly. In some embodiments, the CoQ10 composition is administered orally weekly at a dose of about 5 mg/kg to about 2000 mg/kg. In some embodiments, the CoQ10 composition is administered orally weekly at a dose of about 50 mg/kg to about 2000 mg/kg. In some embodiments, the CoQ10 composition is administered orally weekly at a dose of about 100 mg/kg to about 2000 mg/kg. In some embodiments, the CoQ10 composition is administered orally weekly at a dose of about 500 mg/kg to about 2000 mg/kg. In some embodiments, the CoQ10 composition is administered orally once per week. In some embodiments, the CoQ10 composition is administered orally twice per week. In some embodiments, the CoQ10 composition is administered orally three times per week. In some embodiments, the CoQ10 composition is administered orally four times per week. In some embodiments, the CoQ10 composition is administered orally five times per week. In some embodiments, the CoQ10 composition is administered orally six times per week. In some embodiments, the CoQ10 composition is administered orally seven times per week. Any of the foregoing oral doses and/or dosing schedules can be combined with any parenteral dose and/or dosing schedule provided herein.

In some embodiments, the CoQ10 composition is administered parenterally (e.g., intravenously) in combination with oral administration of any one or more CoQ10 precursors or building blocks. Building blocks of CoQ10 include, but are not limited to, phenylalanine, tyrosine, 4-hydroxyphenylpyruvate, phenylacetate, 3-methoxy-4-hydroxymandelate, vanillic acid, 4-hydroxybenzoate, mevalonic acid, farnesyl, 2,3-dimethoxy-5-methyl-p-benzoquinone, as well as the corresponding acids or ions thereof.

In some embodiments, the methods further comprising administering a CoQ10 supplement to the subject. The additional CoQ10 supplement can be any commercially available CoQ10 formulation. In some embodiments, the methods further comprise administering a CoQ10 supplement orally to the subject. In some embodiments, the CoQ10 supplement is administered orally at a dose of about 5 mg/kg to about 200 mg/kg. In some embodiments, the CoQ10 supplement is administered orally at a dose of about 30 mg/kg. In some embodiments, the CoQ10 supplement is administered orally every day. In some embodiments, the CoQ10 supplement is administered orally every week. In some embodiments, the CoQ10 supplement is administered orally at a dose of about 10 mg/kg to about 100 mg/kg per day. In some embodiments, the CoQ10 supplement is administered orally at a dose of about 10 mg/kg to about 50 mg/kg per day. In some embodiments, the CoQ10 supplement is administered orally at a dose of about 30 mg/kg per day.

In some embodiments, the methods further comprise administering Vitamin K to the subject. Vitamin K is administered to subjects prophylactically in an amount and frequency to reduce coagulation-related adverse events, such as significant prolongation of PT (INR) and PTT intervals. In some embodiments, Vitamin K is administered to the subject at a dose of between about 1 and 50 mg/week, or 5 and 20 mg/week. In some embodiments, Vitamin K is administered to the subject at a dose of at least 10 mg/week. In some embodiments, Vitamin K is administered to the subject at a dose of about 10 mg/week.

In some embodiments, administration, e.g., parenteral (e.g., IV) and/or oral administration, of the CoQ10 composition as described herein increases the plasma concentration of one or more metabolites of quinone metabolism. In some embodiments, the one or more metabolites of quinone metabolism is selected from the group consisting of Coenzyme Q1, Coenzyme Q2, Coenzyme Q4, phylloquinone, menaquinone, menadione, 1,2-naphthoquinone, D-alpha-tocopherylquinone, p-Benzoquinone, duroquinone, idebenone, 2-methoxy-1,4-naphthoquinone, 2,6-dimethoxy-1,4-benzoquinone, adrenochrome, 1,8-dihydroxyanthraquinone, chrysophanol, thymoquinone, 2,6-di-tert-butyl-1,4-benzoquinone, emodin, and pyrroloquinoline quinone. In some embodiments, the one or more metabolites of quinone metabolism is selected from the group consisting of menadione, duroquinone, and 1,8-dihydroxyanthraquinone.

In some embodiments, administration, e.g., parenteral (e.g., IV) and/or oral administration, of the CoQ10 composition as described herein results in a plasma concentration of at least about 0.5 μg/mL to about 300 μg/mL for one or more of the metabolites of quinone metabolism in the subject. In some embodiments, administration, e.g., parenteral (e.g., IV) and/or oral administration, of the CoQ10 composition as described herein results in a plasma concentration of at least about 0.5 μg/mL to about 200 μg/mL for one or more of the metabolites of quinone metabolism in the subject. In some embodiments, administration, e.g., parenteral (e.g., IV) and/or oral administration, of the CoQ10 composition as described herein results in a plasma concentration of at least about 0.5 μg/mL to about 100 μg/mL for one or more of the metabolites of quinone metabolism in the subject. In some embodiments, administration, e.g., parenteral (e.g., IV) and/or oral administration, of the CoQ10 composition as described herein results in a plasma concentration of at least about 100 μg/mL to about 300 μg/mL for one or more of the metabolites of quinone metabolism in the subject. In some embodiments, administration, e.g., parenteral (e.g., IV) and/or oral administration, of the CoQ10 composition as described herein results in a plasma concentration of at least about 0.5 μg/mL to about 50 μg/mL for one or more of the metabolites of quinone metabolism in the subject. In some embodiments, administration, e.g., parenteral (e.g., IV) and/or oral administration, of the CoQ10 composition as described herein results in a plasma concentration of at least about 50 μg/mL to about 100 μg/mL for one or more of the metabolites of quinone metabolism in the subject. In some embodiments, administration, e.g., parenteral (e.g., IV) and/or oral administration, of the CoQ10 composition as described herein results in a plasma concentration of at least about 100 μg/mL to about 150 μg/mL for one or more of the metabolites of quinone metabolism in the subject. In some embodiments, administration, e.g., parenteral (e.g., IV) and/or oral administration, of the CoQ10 composition as described herein results in a plasma concentration of at least about 150 μg/mL to about 200 μg/mL for one or more of the metabolites of quinone metabolism in the subject. In some embodiments, administration, e.g., parenteral (e.g., IV) and/or oral administration, of the CoQ10 composition as described herein results in a plasma concentration of at least about 200 μg/mL to about 250 μg/mL for one or more of the metabolites of quinone metabolism in the subject. In some embodiments, administration, e.g., parenteral (e.g., IV) and/or oral administration, of the CoQ10 composition as described herein results in a plasma concentration of at least about 250 μg/mL to about 300 μg/mL for one or more of the metabolites of quinone metabolism in the subject.

In some embodiments, administration, e.g., parenteral (e.g., IV) and/or oral administration, of the CoQ10 composition as described herein results in a plasma concentration of at least about 0.5 μg/mL, e.g., at least about 1 μg/mL, about 5 μg/mL, about 10 μg/mL, about 15 μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, about 100 μg/mL, about 125 μg/mL, about 150 μg/mL, about 175 μg/mL, about 200 μg/mL, about 250 μg/mL, or about 300 μg/mL for one or more of the metabolites of quinone metabolism in the subject.

### Methods of Detecting and Measuring the Level of CoQ10 and Related Analogs

The level of CoQ10 and related analogs, e.g., metabolites of quinone metabolism, in a biological sample, e.g., a plasma sample, or a tissue sample, e.g., a brain, heart, kidney or muscle tissue, in the methods provided herein can be determined by any methods known in the art.

Blood collected into anti-coagulant can be processed using standard procedure known in the art to extract plasma. In some embodiments, anti-coagulated blood is processed immediately after collection to extract plasma. In some other embodiments, blood is collected into pre-cooled heparinized vials and kept at low temperature until it is processed to extract plasma.

In some embodiments, protein precipitation is further applied to a plasma sample to remove most of the protein from the sample, leaving CoQ10 and its related analogs in the supernatant. The samples may be centrifuged to separate the liquid supernatant from the precipitated proteins; alternatively the samples may be filtered to remove precipitated proteins. The resultant supernatant or filtrate may then be applied directly for analysis.

In some embodiments, total levels of CoQ10 and/or related analogs in plasma are determined using enzyme-linked immunosorbent assay (ELISA) technique, for which kits are commercially available (e.g., Human Coenzyme Q10 (Coq10) Elisa Kit Biomatik™). In a non-limiting example, a microtiter plate is pre-coated with CoQ10, and standards or samples containing CoQ10 are added to the appropriate microtiter plate wells with horseradish-peroxidase (HRP)-conjugated anti-CoQ10 antibody. Pre-coated CoQ10 and CoQ10 in the samples compete for binding to the antibody, After washing and color-development for the bound HRP-anti-CoQ10 antibody, signal from the wells corresponding to the plasma sample is compared to signals from the wells corresponding to the standards to determine the CoQ10 amount in the plasma sample.

In some embodiments, the CoQ10 level in plasma is determined using spectrometry. For example, extracted CoQ10 from a plasma sample is dissolved in an appropriate solvent and analyzed using UV spectroscopic detection of CoQ10; readout of absorption at 275 nm of the sample is compared to a standard curve established by standards with known concentrations of CoQ10 to determine the amount of CoQ10 in the plasma sample.

In some embodiments, the levels of CoQ10 and/or its related analogs in plasma are determined using liquid chromatography. For example, U.S. Pat. No. 7,303,921B2 teaches a method of extracting CoQ10 from a sample and analyze the CoQ10 extract using high pressure liquid chromatography (HPLC). Paredes-Fuentes et al. (Coenzyme Q10 Treatment Monitoring in Different Human Biological Samples, Antioxidants 2020, 9, 979) discloses measuring CoQ10 using HPLC coupled to electro-chemical detection. The entire contents of each of the foregoing references are expressly incorporated herein by reference.

In some embodiments, the levels of CoQ10 and/or its related analogs in plasma are determined using mass spectrometry.

As used herein, the term “mass spectrometry” or “MS” refers to an analytical technique to identify compounds by their mass. MS refers to methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer and an ion detector. In general, one or more molecules of interest are ionized, and the ions are subsequently introduced into a mass spectrometric instrument where, due to a combination of magnetic and electric fields, the ions follow a path in space that is dependent upon mass (“m”) and charge (“z”). See, e.g., U.S. Pat. Nos. 6,204,500, entitled “Mass Spectrometry From Surfaces;” 6,107,623, entitled “Methods and Apparatus for Tandem Mass Spectrometry;” 6,268,144, entitled “DNA Diagnostics Based On Mass Spectrometry;” 6,124,137, entitled “Surface-Enhanced Photolabile Attachment And Release For Desorption And Detection Of Analytes;” Wright et al., Prostate Cancer and Prostatic Diseases 1999, 2:264-76; and Merchant and Weinberger, Electrophoresis 2000, 21:1164-67.

In some embodiments of the methods provided herein, the concentration of CoQ10 in plasma achieved in a subject following administration to the subject of a composition comprising CoQ10 is the concentration of the oxidized form of CoQ10, the concentration of the reduced form of CoQ10, or the concentration of both the oxidized and reduced forms, i.e., the total concentration of CoQ10.

In some embodiments, the oxidized form of CoQ10 and reduced form of CoQ10 (CoQ10H2) can be specifically determined using methods known in the art (see, e.g, Claessens et al. Rapid and sensitive analysis of reduced and oxidized coenzyme Q10 in human plasma by ultra performance liquid chromatography-tandem mass spectrometry and application to studies in healthy human subjects, Ann Clin Biochem. 2016 March; 53 (Pt 2): 265-273, the entire content of which is incorporated herein by reference). Measurement of both oxidized and reduced forms of CoQ10 provides for the total level of CoQ10 in a sample.

In some embodiments, the oxidized and/or reduced forms of CoQ10 is measured using methods such as those disclosed in WO 2013/131068, the entire content of which is incorporated herein by reference. In brief, internal standards for the oxidized and/or reduced form of CoQ10 are generated by creating isotopically labeled analogs of these two forms of CoQ10. In some embodiments, the oxidized and reduced form of CoQ10 used as internal standards are deuterated. A blood or plasma sample containing CoQ10 is spiked with known amount of deuterated oxidized CoQ10 (CoQ10-d6) and/or reduced CoQ10 (CoQ10H2-d6). The spiked sample, which could be further extracted for CoQ10, is subjected to MS analysis for oxidized and/or reduced CoQ10 quantification.

As used herein, an isotopically labeled analog of the oxidized form of coenzyme Q10 (CoQ10-d6) is the compound having the following structural formula:

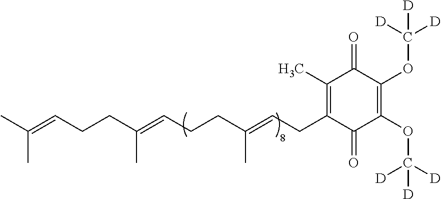

As used herein, an isotopically labeled analog of the reduced form of coenzyme Q10 (CoQ10H₂-d6) is the compound having the following structural formula:

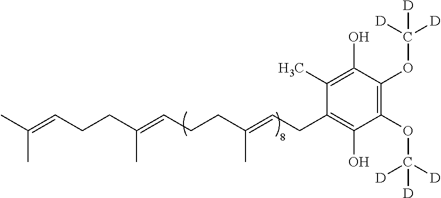

In some embodiments, CoQ10H₂-d6 is synthesized by reacting CoQ10-d6 with one or more reducing agents. In some embodiments, the reducing agent is sodium borohydride (NaBH₄).

As provided herein, the oxidized and reduced forms of CoQ10 in a sample may be detected and quantified separately or they both may be detected and quantified together, in a single analytical run. In a specific embodiment, the methods of the present invention may be used to quantify the amount of reduced CoQ10 (CoQ10H₂) present in a sample. These methods allow accurate and reliable quantification of CoQ10H₂, which is important because CoQ10H₂ has been reported to be the biologically active form of CoQ10. In a specific embodiment, the methods of the present invention may be used to quantify the amount of oxidized CoQ10 present in a sample. These methods allow accurate and reliable quantification of oxidized CoQ10, which is important because oxidized CoQ10 has been reported to be an important biologically active form of CoQ10 for treating certain conditions, e.g., cancer and cardiovascular disease.

In some embodiments, the methods of detection and quantification of oxidized and reduced forms of CoQ10 may be combined with the sample extraction methods described herein or with other extraction methods. For example, in some embodiments, the reduced and oxidized forms of CoQ10 in a sample, e.g., a biological sample, may be detected and quantified by extracting the sample using one extraction buffer, e.g., 1-propanol. In other embodiments, the reduced and oxidized forms of CoQ10 in a sample, e.g., a biological sample, may be detected and quantified by extracting the sample using a first and a second extraction buffer according to the methods described herein.

Quantification of the oxidized and reduced forms of CoQ10 is accomplished by using their respective isotopically labeled versions as internal standards, e.g., oxidized and reduced deuterated COQ10. In some embodiments, CoQ10-d6 is used as an internal standard for determining the amount of CoQ10 in a sample. In some embodiments, CoQ10H₂-d6 is used as an internal standard for determining the amount of CoQ10H₂ in a sample. In some embodiments, CoQ10-d6 and CoQ10H₂-d6 are both added to the sample to be used for simultaneous determination of the amounts of oxidized CoQ10 and CoQ10H₂ contained in the sample. The amount of oxidized CoQ10 in a sample can be calculated based on the known amount of CoQ10-d6 added to the sample and on the relative mass spectrometric signals produced by oxidized CoQ10 and CoQ10-d6. Similarly, the amount of CoQ10H₂ in a sample can be calculated based on the known amount of CoQ10H₂-d6 added to the sample and on the relative mass spectrometric signals produced by CoQ10H₂ and CoQ10H₂-d6.

The use of CoQ10-d6 as an internal standard for quantifying oxidized CoQ10 in a sample, and the use of CoQ10H₂-d6 for quantifying CoQ10H₂ in a sample provides advantages over the known methods that employ CoQ10-d6 or analogs such as CoQ9 or CoQ11, as internal standards for quantifying both oxidized CoQ10 and CoQ10H₂. Specifically, the quantification methods described herein provide greater accuracy of CoQ10H₂ quantification because CoQ10H₂-d6, used in the methods as an internal standard, has properties, e.g., extraction recovery, ionization response and chromatographic retention time that are very similar to those of CoQ10H₂, but also produces different mass spectrometric signals. In contrast, the use of CoQ10-d6, CoQ9 or CoQ11, as has been described in the art, as internal standards for quantifying CoQ10H₂ results in a biased quantification and lower accuracy.

The internal standard, e.g., CoQ10-d6 or CoQ10H₂-d6, can be added to a sample at any point of the sample work-up procedure. In some embodiments, the internal standard is added to each sample in the beginning of sample processing, e.g., extraction. In some embodiments, the sample comprises blood, and the internal standard is added to the sample prior to plasma extraction.

The amount of internal standard, e.g., CoQ10-d6 or CoQ10H₂-d6, to be added to each sample should not interfere with the mass spectrometric signal produced by the analyte, oxidized CoQ10 or CoQ10H₂. In some embodiments, the amount of CoQ10-d6 or CoQ10H₂-d6 to be added to each sample should result in a concentration of CoQ10-d6 or CoQ10H₂-d6 that is within the linear range of the dose response curve. In a preferred embodiment, the amount of CoQ10-d6 or CoQ10H₂-d6 to be added to each sample should produce a concentration between 20 and 2500 ng/mL, (e.g., 50 and 2000 ng/ml, or 100 and 1500 ng/ml or between 100 and 1000 ng/ml).

In certain embodiments, the LC-MS/MS method for determining the amount of oxidized CoQ10 and CoQ10H₂ is linear over the clinically relevant range, e.g., of 20-2500 ng/ml, for both forms, with r²>0.99. The % CV's and inter and intra-assay precision/accuracy values for both forms is below 10%.

In some embodiments, the method of determining the amount of oxidized CoQ10 and/or CoQ10H₂ in a sample comprises:
a) providing the sample;
b) optionally adding a known amount of CoQ10-d6 and/or CoQ10H₂-d6 to the sample;
c) processing the sample;
d) optionally subjecting the sample to liquid chromatography;
e) detecting oxidized CoQ10 and/or CoQ10H₂ and, if applicable, CoQ10-d6 and CoQ10H₂-d6 by mass spectrometry; and
f) determining the amount of detected oxidized CoQ10 and/or CoQ10H₂ in the sample by comparing it to the known amount of detected CoQ10H₂-d6.

In some embodiments, step c may comprise sample extraction using a first extraction buffer and a second extraction buffer.

In some embodiments, the extracted sample may be further subjected to liquid chromatography in step d to separate oxidized CoQ10 and CoQ10H₂ and their isotopically labeled analogs, if applicable. Traditional chromatographic analysis relies on column packing in which laminar flow of the sample through the column is the basis for separation of the analyte of interest from the sample. The skilled artisan will be able to select LC, including HPLC, instruments and columns that are suitable for use with oxidized CoQ10 and CoQ10H₂. The chromatographic column typically includes a medium (i.e., a packing material) to facilitate separation of chemical moieties (i.e., fractionation). The medium may include minute particles, or may include a monolithic material with porous channels. A surface of the medium typically includes a bonded surface that interacts with the various chemical moieties to facilitate separation of the chemical moieties. One suitable bonded surface is a hydrophobic bonded surface such as an alkyl bonded, cyano bonded surface, or highly pure silica surface. Alkyl bonded surfaces may include C-4, C-8, C-12, or C-18 bonded alkyl groups. In preferred embodiments, the column is a C18 microparticle packed column (such as Agilent C18 Zorbax column). The chromatographic column includes an inlet port for receiving a sample and an outlet port for discharging an effluent that includes the fractionated sample.

Any chromatographic method that results in effective separation of oxidized CoQ10 and CoQ10H₂ may be used. In some embodiments, the elution of oxidized CoQ10 and CoQ10H₂ from a reversed phase column is accomplished using an isocratic elution mode, i.e., wherein the composition of the mobile phase is kept constant. In some embodiments, the composition of mobile phase is 30:70 to 90:10 A: B, wherein A is 5 mM ammonium formate and B is 1-propanol. The composition of the mobile phase can be changed by substituting 5 mM ammonium formate and 1-propanol with any other solvents having similar polarity, including, but not limited to ethanol, 2-propanol, acetone or acetonitrile. In a preferred embodiment, the composition of the mobile phase is 80:20 A: B, wherein A is 5 mM ammonium formate and B is 1-propanol, and the chromatographic separation is carried out for 5 minutes.

In some embodiments, oxidized CoQ10 and CoQ10H₂ and their isotopically labeled analogs, if applicable, may be detected during chromatography. In some embodiments, the detection may comprise spectroscopic detection. Oxidized CoQ10 is preferably detected at a wavelength near or at 275 nm (e.g., 270-280 nm; 272-278 nm; 274-276 nm), using ultraviolet spectroscopy.

In preferred embodiments, the eluted oxidized CoQ10 and CoQ10H₂ and their isotopically labeled analogs, if applicable, are fed directly into a mass spectrometer after chromatographic separation. In an alternative embodiments, the chromatographic fraction comprising the eluted oxidized CoQ10 and CoQ10H₂ and their isotopically labeled analogs, if applicable, may first be collected and then introduced into a mass spectrometer in a separate step.

In step e, oxidized CoQ10 and CoQ10H₂ and their isotopically labeled analogs, if applicable, are detected using mass spectrometry. During mass spectrometry, oxidized CoQ10 and CoQ10H₂ may be ionized by any method known to the skilled artisan. Mass spectrometry is performed using a mass spectrometer, which includes an ion source for ionizing the fractionated sample and creating charged molecules for further analysis. For example ionization of the sample may be performed by electron ionization, chemical ionization, electrospray ionization (ESI), photon ionization, atmospheric pressure chemical ionization (APCI), photoionization, atmospheric pressure photoionization (APPI), Laser diode thermal desorption (LDTD), fast atom bombardment (FAB), liquid secondary ionization (LSI), matrix assisted laser desorption ionization (MALDI), field ionization, field desorption, thermospray/plasmaspray ionization, surface enhanced laser desorption ionization (SELDI), inductively coupled plasma (ICP) and particle beam ionization. The skilled artisan will understand that the choice of ionization method may be determined based on the analyte to be measured, type of sample, the type of detector, the choice of positive versus negative mode, etc.

Oxidized CoQ10 and CoQ10H₂ and their isotopically labeled analogs may be ionized in positive or negative mode. In a preferred embodiment, oxidized CoQ10 and CoQ10H₂ and their isotopically labeled analogs are ionized using ESI in positive ion mode.

In mass spectrometry techniques generally, after the sample has been ionized, the positively or negatively charged ions thereby created may be analyzed to determine a mass-to-charge ratio. Suitable analyzers for determining mass-to-charge ratios include quadrupole analyzers, ion traps analyzers, and time-of-flight analyzers. Exemplary ion trap methods are described in Bartolucci, et al., Rapid Commun. Mass Spectrom. 2000, 14:967-73.

The ions may be detected using several detection modes. For example, selected ions may be detected, i.e. using a selective ion monitoring mode (SIM), or alternatively, mass transitions resulting from collision induced dissociation or neutral loss may be monitored, e.g., multiple reaction monitoring (MRM) or selected reaction monitoring (SRM). In some embodiments, the mass-to-charge ratio is determined using a quadrupole analyzer. For example, in a “quadrupole” or “quadrupole ion trap” instrument, ions in an oscillating radio frequency field experience a force proportional to the DC potential applied between electrodes, the amplitude of the RF signal, and the mass/charge ratio. The voltage and amplitude may be selected so that only ions having a particular mass/charge ratio travel the length of the quadrupole, while all other ions are deflected. Thus, quadrupole instruments may act as both a “mass filter” and as a “mass detector” for the ions injected into the instrument.

One may enhance the resolution of the MS technique by employing “tandem mass spectrometry,” or “MS/MS”. In this technique, a precursor ion (also called a parent ion) generated from a molecule of interest can be filtered in an MS instrument, and the precursor ion subsequently fragmented to yield one or more fragment ions (also called daughter ions or product ions) that are then analyzed in a second MS procedure. By careful selection of precursor ions, only ions produced by certain analytes are passed to the fragmentation chamber, where collisions with atoms of an inert gas produce the fragment ions.

### III. CoQ10 Composition For Use in Methods of the Invention

The present invention further provides CoQ10 compositions for use in the methods of the present invention, e.g., methods for treating primary CoQ10 deficiency. The CoQ10 compositions used in the methods of the invention are highly bioavailable, and thereby result in delivery of substantially higher concentrations of CoQ10 in plasma and/or other tissues, e.g., brain, blood, spleen, pancreas, liver and lungs, as compared to that which can be achieved with over-the-counter oral CoQ10 supplements.

The highly bioavailable compositions comprising CoQ10 suitable for use in the methods provided herein include, for example, compositions described in PCT/US2011/028042, the entire contents of which are incorporated herein by reference.

In some embodiments, the CoQ10 composition is formulated as a nano-dispersion. In some embodiments, the CoQ10 composition comprises an aqueous solution; a CoQ10 dispersed into a colloidal nano-dispersion of particles; and at least one of a dispersion stabilizing agent and an opsonization reducer; wherein the nano-dispersion of the CoQ10 is dispersed into nano-particles having a mean particle size of less than 200-nm

Through high pressure homogenization, CoQ10 particles are reduced to produce particles that are small enough to pass through a 200-nm sterilizing filter. Particles that are small enough to pass through a 200-nm sterilizing filter can be injected intravenously. These particles are much smaller than blood cells and therefore will not embolize capillaries. Red blood cells for example are 6-mm×2-mm disks. The particles are dispersed to and are encased or surrounded by a stabilizing agent. While not wishing to be bound by any theory, it is believed that the stabilizing agents are attracted to CoQ10 such that the dispersed particles of CoQ10 are surrounded by the stabilizing agent forming a suspension or an emulsion. The dispersed particles in the suspension or emulsion comprises a stabilizing agent surface and a core consisting of CoQ10 in a solid particulate form (suspension) or in an immiscible liquid form (emulsion). In certain aspects, the dispersed particles are entrenched in the lipophilic regions of a liposome.

The dispersed colloidal system provided herein provides certain performance advantages over the prior art. For example, the present invention permits a high drug load in the composition without the use of co-solvents. Additionally, high and relatively reproducible plasma levels are achieved without the dependence on endogenous low-density lipoprotein carriers. More importantly, the present invention allows sustained high drug levels in solid tumors due to the passive accumulation of the colloidal particles of CoQ10.

The present intravenous formulation substantially comprises a continuous phase of water and dispersed solids (suspension) or dispersed immiscible liquid (emulsion). Dispersed colloidal systems, in which the particles are composed largely of the active agent (CoQ10) itself, can often deliver more drug per unit volume than continuous solubilizing systems, if the system can be made adequately stable. The present invention provides colloidal dispersions of poorly water-soluble active agents, such as CoQ10.

By utilizing mechanical devices, such as a microfluidizer, the particle size is reduced by high pressure continuous homogenization, forming colloidal-sized droplets in a spray system, or by shearing the particles in a liquid flowing at high velocity in a restricted and tortuous passage.

Significant energy is required to cleave the bulk particle itself. The smaller particles increases the interfacial area of the active agent. Surfactants are used to reduce the interfacial energy thereby stabilizing the dispersion. The particle size determines the total interfacial area and the interfacial energy that must be accommodated to achieve a stable system. As the particle size goes down, more energy is required to produce the particle and since the total surface area goes up, the surfactant must accommodate a greater interfacial energy.

Through high pressure homogenization, the particle size of CoQ10 is reduced to less than 200-nm. In some embodiments, the particle size is reduced to between 10-nm and 200-nm or between 10-nm and 100-nm or more preferably between 30-nm and 80-nm. In some embodiments, the resulting colloidal CoQ10 particles are in an amorphous super-cooled state as defined herein.

In certain embodiments, the dispersed CoQ10 particles are crystallized by a lyophilization process to produce nano-dispersion particles wherein the active agent core was in crystalline form. Polarizing light microscopy (PLM) or X-ray powder diffraction (XRDP) was used to confirm the crystalinity of the CoQ10 colloidal dispersion and compared to the XRDP of bulk CoQ10. In form R, a formulation comprising 5.0 wt % CoQ10, 3.3 wt % poloxamer and 91 wt % of water, was cycled 20 times in a microfluidizer and then lyophilized to produce the particles. The XRDP demonstrates that the CoQ10 particles were crystalline. In form A, a formulation comprising 3 wt % CoQ10, 1.8 wt % Lipoid SPC-3 and 95.2 wt % water, was cycled 20 times in a microfluidizer to reduce the particle size. The particles were then lyophilized to produce the particles. The XRDP demonstrates that the CoQ10 particles were crystalline. In form O, a formulation comprising 5.0 wt % CoQ10, 3.0 wt % DMPC and 92 wt % of water, was cycled 20 times in a microfluidizer and then lyophilized to produce the particles. The XRDP demonstrates that the CoQ10 particles were crystalline. In form C, a formulation comprising 5.0 wt % CoQ10, 3.3 wt % poloxamer and 91 wt % of water, was cycled 20 times in a microfluidizer and then lyophilized to produce the particles. The XRDP demonstrates that the CoQ10 particles were crystalline. In form G, a formulation comprising 5.0 wt % CoQ10, 3.0 wt % Lipoid SPC-3 and 92 wt % of water, was cycled 20 times in a microfluidizer and then lyophilized to produce the particles. The XRDP demonstrates that the CoQ10 particles were crystalline. In form Q, a formulation comprising 5.0 wt % CoQ10, 2.5 wt % DMPC, 0.5 wt % sodium deoxycholate and 92 wt % of water, was cycled 20 times in a microfluidizer and then lyophilized to produce the particles. The XRDP demonstrates that the CoQ10 particles were crystalline. In form S, a formulation comprising 7.5 wt % CoQ10, 4.5 wt % DMPC and 88 wt % of water, was cycled 20 times in a microfluidizer and then lyophilized to produce the particles. The XRDP demonstrates that the CoQ10 particles were crystalline. In form T, a formulation comprising 7.5 wt % CoQ10, 5.0 wt % polaxamer and 87.5 wt % of water, was cycled 20 times in a microfluidizer and then lyophilized to produce the particles. The XRDP demonstrates that the CoQ10 particles were crystalline. In form U, a formulation comprising 7.5 wt % CoQ10, 4.0 wt % DMPC, 1.0 wt % poloxamer 188 and 87.5 wt % of water, was cycled 20 times in a microfluidizer and then lyophilized to produce the particles. The XRDP demonstrates that the CoQ10 particles were crystalline. In form V, a formulation comprising 3.0 wt % CoQ10, 1.5 wt % DMPC and 95.5 wt % of water, was cycled 20 times in a microfluidizer and then lyophilized to produce the particles. The XRDP demonstrates that the CoQ10 particles were crystalline.

In lyophilizing the particles, the dryer was cooled to −35° C. Three milliliters of each of the above formulation was added to the 5 mL serum vial, in duplicate. Serum stopper was placed on top but allowed room for water vapor to escape. Formulations were placed in −78° C. freezer for 1-hour to rapid freeze. After this period, all were transferred in toto to the middle shelf of dryer. Vacuum was immediately initiated. After 16 hours the temperature was adjusted from −35° C. to −30° C. After 24 hours the temperature was adjusted from −30° C. to −28° C. After 2 hours, the temperature was adjusted from −28° C. to −26° C. After 4 hours the temperature was further adjusted to between −26° C. and−25° C. After reaching−25° C., the vials were stoppered and the vacuum released to ambient air.

In reducing the dispersion particle size, it may be desirable for the CoQ10 mixture to go through several passes through a Microfluidizer to obtain the desired particle size. After a single pass through M110P Microfluidizer with F12Y interaction chamber with 75-μm passages, particles of less than 200-nm mean diameter were produced. After 20 passes the mean diameter of the particles were less than 50-nm. The formulation contained 5g of CoQ10, 3g of DMPC and 92 mL of water. One of ordinary skill in the art will understand that the amounts of the CoQ10, DMPC and water may be adjusted depending on the desired therapeutic use. The Microfluidizer operated at a maximum pressure of 25,000 PSI. In certain embodiments, it is preferable to add at least one of a dispersion stabilizing agent and an opsonization reducer. In certain embodiments, the colloidal particles are prepared using both the dispersion stabilizer agent and the opsonization reducer. Preferred dispersion stabilizing agents include Polyethoxylated (a/k/a pegylated) castor oil (Cremophor® EL), Polyethoxylated hydrogenated castor oil (Cremophor® RH 40), Tocopherol polyethylene glycol succinate (Pegylated vitamin E, Vitamin E TPGS), Polysorbates (Tweens®), Sorbitan fatty acid esters (Spans®), Bile acids and bile-acid salts and DMPC while preferred opsonization reducers include Polyethylene glycol of various chain lengths, polysaccharides, other PEG-containing copolymers, poloxamines or poloxamers such as poloxamer 188. In certain embodiments, heparin also constitutes a suitable opsonization reducer. The poloxamer provides a hydrophilic surface so as to reduce particle opsonization after administration. The poloxamer also functions as a particle surface modifier, to add bulky chains to reduce opsonization by steric interaction. Poloxamer 188 (Pluronic® F68, Lutrol® F68) has approximately 28 PPG units in the center block and 79 PEG units in the end blocks. The hydrophobic center block anchors the molecule to the particle, and the PEG end blocks are extended from the particle. Opsonization is reduced by both the hydrophilicity of the PEG chains and by the steric (space-filling) effects of the chains (i.e., proteins can't get to the surface).

Through the methods, further described herein, the present invention provides a therapeutic formulation that is suitable for administration to a subject. The therapeutic formulation includes an aqueous solution. In certain embodiments of the present invention, the aqueous solution is water. The aqueous solution may function as either or both the dispersion medium for the colloidal system or as the formulation medium for parenteral administration and delivery of the colloidal particles. As the dispersion medium, the aqueous solution may contain other water soluble or dispersible stabilizers, isotonicity agents such as glycerol or xylitol, lyoprotectants such as sucrose, glucose, trehalose, etc., electrolytes, buffers, antilloculants such as sodium citrate, sodium pyrophosphate or sodium dodecylsulfate or preservatives.

Lyoprotectants comprise but are not limited to the group consisting of sugars, polyols (such as e.g. sugar alcohols) and amino acids. Preferred lyoprotectants include sugars such as sucrose, trehalose, and glucose. Other suitable sugars include, lactose, mannose, maltose, galactose, fructose, sorbose, raffinose, neuraminic acid, amino sugars such as glucosamine, galactosamine, N-methylglucosamine (“Meglumine”), polyols such as mannitol and sorbitol, and amino acids such as arginine and glycine.

As the formulation medium, the aqueous solution may include Hank's solution, ringer's solution, phosphate buffered saline (PBS), physiological saline buffer or other suitable salts or combinations to achieve the appropriate pH and osmolarity for parenterally delivered formulations. The aqueous solution may contain substances which increase the viscosity of the solution, such as sodium carboxymethyl cellulose, sorbitol, or dextran.

In some embodiments, CoQ10 is dispersed in the aqueous solution such that a colloidal dispersion is formed wherein the nano-dispersion particles of CoQ10 are covered or encased or encircled by the dispersion stabilizing agents to form nano-dispersions of CoQ10 particles. The nano-dispersed CoQ10 particles have a core formed of CoQ10 that is surrounded by the stabilizing agent. Similarly, in certain aspects, the stabilizing agent is a phospholipid having both a hydrophilic and lipophilic portion. The phospholipids form liposomes or other nanoparticles upon homogenization. In certain embodiments, these liposomes are bi-layered unilamellar liposomes while, in other embodiments, the liposomes are bi-layered multi-lamellar liposomes. The dispersed CoQ10 particles are dispersed in the lipophilic portion of the bi-layered structure of the liposome formed from the phospholipids. In certain embodiments, the core of the liposome, like the core of the nano-dispersion of CoQ10 particles is formed of CoQ10 and the outer layer is formed of the bi-layered structure of the phospholipid. In certain embodiments, the colloidal dispersions are treated by a lyophilization process whereby the nanoparticle dispersion is converted to a dry powder.

Coenzyme Q10, also referred to herein as CoQ10, is also known as ubiquinone, or ubidecarenone. CoQ10 is art-recognized and further described in International Publication No. WO 2005/069916, the entire disclosure of which is incorporated by reference herein. CoQ10 is one of a series of polyprenyl 2,3-dimethoxy-5-methylbenzoquinone (ubiquinone) present in the mitochondrial electron transport systems of eukaryotic cells. Human cells produce CoQ10 exclusively and it is found in cell and mitochondrial membranes of all human cells, with the highest levels in organs with high energy requirements, such as the liver and the heart. The body pool of CoQ10 has been estimated to be about 2 grams, of which more than 50% is endogenous. Approximately 0.5 grams of CoQ10 is required from the diet or biosynthesis each day. CoQ10 is produced in ton quantities from the worldwide supplement market and can be obtained from Kaneka, with plants in Pasadena, Texas and Takasagoshi, Japan.

The tissue distribution and redox state of CoQ10 in humans has been characterized. Typically, tissues with high-energy requirements or metabolic activity such as the heart, kidney, liver and muscle have relatively high concentrations of CoQ10. Most of the CoQ10 in plasma is present as the reduced ubiquinol. A substantial portion of CoQ10 in tissues is in the reduced form as the hydroquinone or ubiquinol. The exception is brain and lung. The oxidized state is presumed to be a reflection of the increased oxidative stress in the tissues. More specifically, in heart, kidney, liver, muscle, intestine and blood (plasma), about 61%, 75%, 95%, 65%, 95% and 96%, respectively, of CoQ10 is in the reduced form.

CoQ10 is very lipophilic and, for the most part, insoluble in water. Due to its insolubility in water, limited solubility in lipids, and relatively large molecular weight, the efficiency of absorption of orally administered CoQ10 is poor. Bhagavan, et al. (Free Rad. Res. 40:445-453 (2006)) reported that that in rats only about 2-3% of orally-administered CoQ10 was absorbed and that CoQ10 is reduced to ubiquinol either during or following absorption in the intestine. In a study by Matthews et al., (Proc. Natl. Acad. Sci. USA 95:8892-8897 (1998)), CoQ10 uptake was found to be age dependent in some tissues. For example, in young rats, plasma, liver, and spleen concentrations increased after four days of dosing, but no increase was observed in heart or kidney. Similarly, oral administration did not increase concentration of CoQ10 in brain in 1-2 month old animals. However, administration of CoQ10 to 12 month old and 24 month old rats resulted in accumulation of CoQ10 in cerebral tissues in both the oxidized and reduced forms. Interestingly, CoQ10 production is stimulated in young rats, but not old rats, after ischemia-reperfusion injury.

In one embodiment of the invention, the hydrophobic bioactive agent is CoQ10, a metabolite of CoQ10, a building block of CoQ10, an analog of CoQ10, or a derivative of CoQ10. An analog of CoQ10 includes analogs having no or at least one isoprenyl repeats. CoQ10 has the following structure:

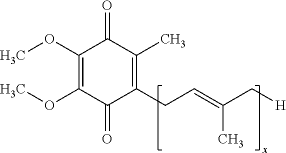

wherein x is 10. In the instant invention, CoQ10 can include derivatives of CoQ10 in which x is any number of isoprenyl units from 4-10, or any number of isoprenyl units from 6-10, or any number of isoprenyl units from 8-10, or 9-10 isoprenyl units. CoQ10 includes the fully oxidized version, also known as ubiquinone, the partially oxidized version, also known as semiquinone or ubisemiquinone, or the fully reduced version, also known as ubiquinol; or any mixtures or combinations thereof.

Building blocks of CoQ10 include any components or synthetic precursors, preferably biologically relevant precursors, of CoQ10. Thus, building blocks of CoQ10 include, but are not limited to, phenylalanine, tyrosine, 4-hydroxyphenylpyruvate, phenylacetate, 3-methoxy-4-hydroxymandelate, vanillic acid, 4-hydroxybenzoate, mevalonic acid, farnesyl, 2,3-dimethoxy-5-methyl-p-benzoquinone, as well as the corresponding acids or ions thereof. Experimental data indicate that building blocks of CoQ10 biosynthesis, such as the precursors for the biosynthesis of the benzoquinone ring, and those for the biosynthesis of the isoprenoid repeats and their attachment to the benzoquinone ring, can be individually administered or administered in combination to target cells to modulate expression of the apoptosis inhibitor Bcl-2 and/or expression of the apoptosis promoter Caspase-3. See, e.g., U.S. patent application Ser. No. 12/778,094 and the examples provided herein.

A metabolite of CoQ10 includes any known metabolite of CoQ10. See e.g., Turunen, M. et al. Biochemica et Biophysica Acta 1660:171-199 (2004), the entire contents of which are incorporated by reference herein. The main metabolite has been identified has an aromatic ring with a side chain shortened to 5-7 atoms. Such a metabolite is shown below. The metabolite may optionally be phosphorylated at carbon 4 or carbon 1.

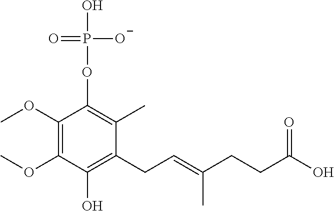 [SMILES: COc1c(O)c(C/C=C(\C)CCC(=O)O)c(C)c(OP(=O)([O-])O)c1OC]

A derivative of CoQ10 includes any compound that is structurally identical to CoQ10, except that one atom is replaced with another atom or group of atoms.

CoQ10 can be located in the core of the colloidal particles where they are dissolved, solubilized or dispersed in the matrix, and/or in the stabilizer layer(s) surrounding the particle core, and/or can be adsorbed to the surface of the colloidal particles. CoQ10 can be dissolved or crystalline or amorphous or a mixture of any of these states. The therapeutic formulation also includes at least one of a dispersion stabilizing agent and an opsonization reducer. The colloidal particles may be liposomes as described herein and may also contain other active agents or other inactive agents, or other hydrophobic or hydrophilic agents.

Dispersion of CoQ10 bulk material into nano-particles increases the interfacial energy as the size of the particles is reduced over passes through the homogenization process. The affinity of a dispersion stabilizing agent such as, for example, DMPC, to the active agent, e.g., CoQ10, nano-particles, causes the dispersion stabilizing agent (e.g., DMPC) to encase the nano-particles and form an CoQ10 nano-dispersion. The dispersion stabilizing agent stabilizes the CoQ10 nano-dispersion by accommodating the high interfacial energy and thereby preventing or reducing coalescence of the dispersed CoQ10 particles. In certain embodiments, liposomes are formed by the colloidal dispersions wherein the phospholipid stabilizer forms a bi-layered system about the dispersed particles of the hydrophobic CoQ10. In certain embodiments, the liposomes are bi-layered unilamellar liposomes. In other embodiments, the liposomes are bi-layered multilamellar liposomes. In certain embodiments, the dispersed particles of the hydrophobic CoQ10 are within lipophilic portion of the bi-layers. In certain other embodiments, where the liposome are multi-lamellar, the hydrophobic CoQ10 is within the lipophilic portion of the bi-layers. In certain other embodiments, where the liposomes are multi-lamellar, the dispersed hydrophobic CoQ10 is within the lipophilic portion of the bi-layer of the liposome and a second agent is in the hydrophilic portion that is between the bi-layered portions of the multi-lamellar liposome.

Proper selection of a surfactant, or a mixture of surfactants, can produce a formulation in which the shelf product is a concentrated solution of drug in liquid surfactants, and upon addition of infusion fluid, the interfacial energy reduction achieved by the surfactants is sufficient to emulsify the system to a colloidal system. The dispersion stabilizing agent may be selected from Polyethoxylated (a/k/a pegylated) castor oil (Cremophor® EL), Polyethoxylated hydrogenated castor oil (Cremophor® RH 40), Tocopherol polyethylene glycol succinate (Pegylated vitamin E, Vitamin E TPGS), Polysorbates (Tweens®), Sorbitan fatty acid esters (Spans®), Bile acids and bile-acid salts and dimyristoylphosphatidyl choline (DMPC). The dispersion stabilizing agent organizes at the interface of the reduced size particles and reduce the interfacial energy, making the dispersion more stable.

Phospholipids have a high affinity for CoQ10, as is demonstrated by the close association of the two in biological membranes. The dispersion stabilizing agent is included in the formulation to, at least, reduce the interfacial tension as the particle size is reduced. In the colloidal dispersion, the nano-dispersion particles include an active agent core surrounded by the stabilizing agent. The dispersion stabilizing agent is typically an amphiphilic substance, i.e. those with a hydrophilic and hydrophobic part of the molecules. At the particle surface, the amphiphilic substances are predominantly arranged in such a way that the hydrophobic part of the molecule protrudes into the core and the hydrophilic part into the surrounding dispersion medium. The surfaces are therefore hydrophilic.

Other suitable phospholipids include lecithin, lysolecithin, phosphatidylcholine, phosphatidylethanolamine, phosphatidylinositol, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, and combinations thereof and therewith.

In one embodiment, the dispersion stabilizing agent is not an agent selected from the group of lecithin, polysorbate 80 and olacta. In one embodiment, the dispersion stabilizing agent is not lecithin. In one embodiment, the dispersion stabilizing agent is not polysorbate 80. In one embodiment, the dispersion stabilizing agent is not olacta.

The formulations of the invention may further include an opsonization reducer. The opsonization reducer may be selected from Polyethylene glycol of various chain lengths, polysaccharides, other PEG-containing copolymers, poloxamines or poloxamers such as poloxamer 188. As defined herein, an opsonization reducer refers to any inactive agent that works in conjunction with the active agent to reduce the ability of opsonins to act as a binding enhancer for the process of phagocytosis. The inactive agent must be included in the FDA's Inactive Ingredient List, which is hereby incorporated by reference in its entirety. The inactive agents must not include pegylated nonionic surfactants (e.g., polysorbate 80, polyethoxylated castor oil, and PEG ethers and esters of fatty alcohols and acids, respectively), since these materials can cause extreme hypersensitivity reactions. Accordingly, in one embodiment, the opsonization reducer is not polysorbate 80. In one embodiment, the opsonization reducer is not polyethoxylated castor oil. In one embodiment, the opsonization reducer is not PEG ethers of fatty alcohols. In one embodiment, the opsonization reducer is not PEG esters of fatty acids.

Colloidal-sized particles larger than 10-nm, for example, are not filtered by the kidneys and will circulate until they are cleared by active processes or they extravasate by diffusing through gaps between vascular endothelial cells. Phagocytic cells of the reticuloendothelial system (RES) or mononuclear phagocytic system (MPS) will capture colloidal particles by endocytosis. These cells include macrophages related to liver (Kupffer cells), spleen, lymph nodes (perivascular macrophages), nervous system (microglia), and bones (osteoclasts). Nonspecific attachment of opsonins (e.g., immunoglobulins, complement components, other serum proteins) marks the particles as foreign. Enzymes and an oxidative-reactive environment in the endosome will destroy the captured particles.

Opsonization of colloidal particles can be reduced, resulting in longer circulation, by a number of factors, including particle size below 100-nm, a neutral or negative surface charge, and adsorption or bonding of bulky hydrophilic chains. An important element of the utility of colloidal drug delivery to solid tumors results from unique anatomical and physiological characteristics of tumors. The capillary network of tumors is tortuous with wide interendothelial junctions (100 to 780-nm) and the tumor has no lymphatic drainage. These characteristics result in passive targeting of colloidal particles to tumors. Particles extravasate through the leaky junctions and remain in the tumor interstitium.

The opsonization reducer is included in the formulation to modify the biological response to the particles. The present invention provides a method wherein the ability to clear the colloidal drug particles by opsonization is reduced by the inclusion of an opsonization reducer in the formulation presented herein. The inclusion of an opsonization reducer results in higher drug levels in tumors than in the plasma.

In one embodiment, the formulation of the invention does not comprise polysorbate 80. In one embodiment, the formulation of the invention does not comprise polyethoxylated castor oil. In one embodiment, the formulation of the invention does not comprise PEG ethers of fatty alcohols. In one embodiment, the formulation of the invention does not comprise PEG esters of fatty acids. In one embodiment, the formulation of the invention does not comprise an agent selected from the group of lecithin, polysorbate 80 and olacta. In one embodiment, the formulation of the invention does not comprise lecithin. In one embodiment, the formulation of the invention does not comprise polysorbate 80. In one embodiment, the formulation of the invention does not comprise olacta.

It has been found, and herein disclosed, that the ratio of the active agent and the inactive agents are important to the control of the particles' size. In order to obtain the benefits of the dispersion stabilizing agent and the opsonization reducer without negatively impacting the benefits of either, or that of the particle size, the ratio of active agent (e.g., CoQ10), dispersion stabilizing agent (e.g., DMPC) and opsonization reducer (e.g., poloxamer) may be adjusted to accommodate a desired particle size and a desired biological response to the colloidal dispersion upon intravenous administration. In certain embodiments, the formulation is prepared such that the weight-per-volume of active agent (e.g., CoQ10), dispersion stabilizing agent (e.g., DMPC) and opsonization reducer (e.g., poloxamer) are each 4%, 3%, and 1.5%, respectively. In certain other embodiments the weight-per-volume of active agent (e.g., CoQ10), dispersion stabilizing agent (e.g., DMPC) and opsonization reducer (e.g., poloxamer) are 8%, 6% and 3.0%, respectively. In certain embodiments, the formulation is prepared such that the weight-per-volume of CoQ10, DMPC and poloxamer are each 4%, 3%, and 1.5%, respectively. In certain other embodiments the weight-per-volume of CoQ10, DMPC and poloxamer are 8%, 6% and 3.0%, respectively.

The hydrophobic bioactive agent, CoQ10, is dispersed at a temperature above its melting point to facilitate dispersion. CoQ10 has a melting point of approximately 48° C. It is herein contemplated that the melting point may vary and may, for example include any value ranging from 47.5° C. to 49.5° C., e.g., 47.5° C., 48.0° C., 48.5° C., 49.0° C. or 49.5° C. In certain embodiments, CoQ10 is mixed in a water bath of 65° C. to form a CoQ10/water mixture, thereby improving the ability to disperse and reduce the particle size of CoQ10.

In some embodiments, CoQ10 is processed through a high-pressure homogenizer (Microfluidizer), such as those available from Microfluidics, Inc. A process stream containing CoQ10 is pumped into an interaction chamber at high velocity and the particles are sheared by wall collisions and cavitations. These shear effects reduce the particle size over repeated passes through the Microfluidizer. The particles of the present invention have size distributions in the nanometer size range with mean particle diameters less than about 200-nm as determined by photon correlation spectroscopy. In one embodiment, the mean size of the nano-dispersion particle is less than about 150-nm. In one embodiment, the mean size of the nano-dispersion particle is less than about 125-nm. In one embodiment, the mean size of the nano-dispersion particle is less than about 100-nm. In one embodiment, the mean size of the nano-dispersion particle is less than about 95-nm, less than about 90-nm, less than about 85-nm, less than about 80-nm, less than about 75-nm, less than about 70-nm, less than about 65-nm, less than about 60-nm, less than about 55-nm, less than about 50-nm, less than about 45-nm, less than about 40-nm, less than about 35-nm, less than about 30-nm, or less than about 25-nm. In one embodiment, the mean size of the nano-dispersion particle is less than about 49-nm, less than about 48-nm, less than about 47-nm, less than about 46-nm, less than about 45-nm, less than about 44-nm, less than about 43-nm, less than about 42-nm, or less than about 41-nm. In one embodiment, the mean size of the nano-dispersion particle is less than about 45-nm. It should be understood that ranges having any one of these values as the upper or lower limits are also intended to be part of this invention, e.g. between about 40-nm and 49-nm, between about 25-nm and 48-nm, or between 25-nm and 47-nm.

In certain other embodiments, through several passes through the Microfluidizer, the mean particle size is reduced to between 10-nm and 200-nm. In one embodiment, the mean particle size is reduced to between 10-nm and 150-nm. In one embodiment, the mean particle size is reduced to between 10-nm and 125-nm. In other embodiments the mean particle size is reduced to between 10-nm and 100-nm. In certain other embodiments the mean particle size is reduced to between 10-nm and 90-nm, between 10-nm and 80-nm, between 10-nm and 70-nm, between 10-nm and 60-nm, between 10-nm and 50-nm, between 10-nm and 45-nm, between 10-nm and 40-nm, or between 10-nm and 30-nm. In certain preferred embodiments the mena particle size is reduced to between 20-nm and 80-nm. In one embodiment, the mean particle size is reduced to between 20-nm and 70-nm. In one embodiment, the mena particle size is reduced to between 20-nm and 60-nm. In one embodiment, the mean particle size is reduced to between 20-nm and 50-nm. In one embodiment, the mean particle size is reduced to between 25-nm and 45-nm. In one embodiment, the mean particle size is reduced to between 30-nm and 45-nm. In certain other preferred embodiments the mean particle size is reduced to between 35-nm and 40-nm. It should be understood that additional ranges having any one of the foregoing values as the upper or lower limits are also intended to be part of this invention, e.g., between 30-nm and 80-nm, or between 30-nm and 40-nm.

It may be preferable to have the colloidal dispersion in the form of a suspension or, alternatively, in the form of an emulsion. As defined elsewhere herein, a suspension, or nanosuspension, comprises a continuous phase and dispersed solids while an emulsion includes a dispersed immiscible liquid. In certain aspects the emulsion includes a dispersed hydrophobic agent, CoQ10, that has been melted and dispersed in a continuous phase to form nano-particles. The melted and dispersed particles may be further dispersed and the size of the particles reduced further by subsequent passes through the homogenization process. As with the solid particles, the smaller the particles of the melted CoQ10, the higher the interfacial energy. A stabilizing agent, such as DMPC, is used to stabilize the dispersed particles by forming a surface layer around the dispersed particles thereby creating nano-dispersed CoQ10 particles. The particles formed are less than 200 nm. The suspension includes particles of the bulk CoQ10 that are dispersed by high energy homogenization. The nano-dispersion of CoQ10, for example, includes dispersed particles of the bulk CoQ10 that are surrounded by a stabilizing agent, such as DMPC. The stabilizing agent forms a surface layer around the dispersed bulk CoQ10 and the dispersed particle of the CoQ10 forms the core of the nano-dispersed particles. In some embodiments the nano-dispersed particles are in an amorphous state. In certain other embodiments, the particles are lyophilized and the CoQ10 core of the nano-dispersion particles of CoQ10 is crystallized.

In some embodiments of the present disclosure, the composition comprising CoQ10 for use in the methods provided herein is an exemplary composition, referred to herein as “BPM31510”, which comprises an aqueous solution, coenzyme Q10, dimyristoylphosphatidyl choline, and poloxamer 188; wherein the coenzyme Q10 is dispersed into a colloidal nano-dispersion of particles having a mean size of less than 50 nm; wherein the coenzyme Q10 is present in said formulation in an amount of 4 wt %; wherein the dimyristoylphosphatidyl choline is present in said formulation in an amount of 3 wt %; and wherein the poloxamer 188 is present in said formulation in an amount of: 1.5 wt %. The composition is suitable for parenteral, e.g., intravenous administration. In some embodiments, the composition is also suitable for oral administration. In some embodiments the coenzyme Q10 is present in the composition in the oxidized form, e.g., at least about 90%, 91%, 92%, 93%, 94%, 95,%, 96%, 97%, 98%, or 99% in the oxidized from.

In some embodiments, the colloidal nano-dispersion is a suspension or an emulsion. In some embodiments, the coenzyme Q10 of the colloidal nano-dispersion is in a crystalline form. In some embodiments, the coenzyme Q10 of the colloidal nano-dispersion is in a super-cooled melt form. In some embodiments, the mean size of the nano-dispersion particles is between 35-nm and 40-nm. In some embodiments, the mean size of the nano-dispersion particles is less than 45-nm.

In some embodiments of the present disclosure, the composition comprising CoQ10 comprises an aqueous solution, coenzyme Q10; dimyristoylphosphatidyl choline, and poloxamer 188; wherein the coenzyme Q10 is dispersed into a colloidal nano-dispersion of particles having a mean size of less than 50 nm, wherein the coenzyme Q10 is present in said formulation in an amount of 4 wt %; wherein the dimyristoylphosphatidyl choline is present in said formulation in an amount of 3 wt %; and wherein the ratio of the coenzyme Q10, the dimyristoylphosphatidyl choline and the poloxamer 188 is 4:3:0.5-1.5, respectively.

In some embodiments the coenzyme Q10 is present in the compositions provided herein in the oxidized form, e.g., at least about 90%, 91%, 92%, 93%, 94%, 95,%, 96%, 97%, 98%, or 99% of the CoQ10 is in the oxidized from. In some embodiments the coenzyme Q10 is present in the compositions provided herein as a mixture of the oxidized and reduced forms. In some embodiments the coenzyme Q10 is present in the compositions provided herein in the reduced form.

It is to be understood that this invention is not limited to particular assay methods, or test agents and experimental conditions described, as such methods and agents may vary. It is also to be understood that the terminology used herein is for the purpose of describing particular embodiments only, and is not intended to be limiting.

The present invention is further illustrated by the following examples, which are not intended to be limiting in any way. The entire contents of all references, patents and published patent applications cited throughout this application, as well as the Figures, are hereby incorporated herein by reference.

### EXAMPLE

### Example 1. Administration of BPM31510 Resulted in High Circulating Levels After IV Infusion

Patients were administered intravenously twice weekly with 72-hour doses of BPM31510 (n=6 at 110 mg/kg dose, for total weekly dose of 220 mg/kg/wk; n=1 at 137 mg/kg dose, for total weekly dose of 274 mg/kg/wk; n=5 at 171 mg/kg dose, for total weekly dose of 342 mg/kg/week) and vitamin K prophylaxis (10 mg/wk). Plasma levels of CoQ in the oxidized form were determined using a mass spectrometry based method as described herein and as described in PCT/US2013/028764, the entire contents of which are incorporated herein by reference.

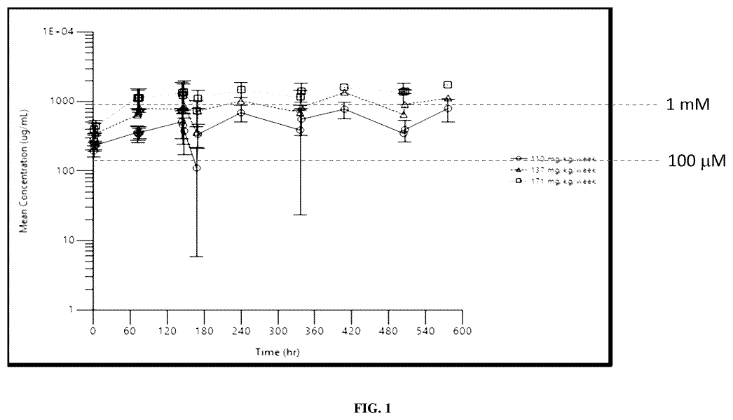

As shown in FIG. 1, IV infusion of BPM31510 achieved significantly high circulating (plasma) levels of CoQ10.

In separate pre-clinical studies in rats, BPM31510 comprising radiolabeled CoQ10 was administered intravenously with a single dose of 100 mg/kg to the animals, and the amount of CoQ10 in various tissues was measured one hour post dosing. The results showed that substantial concentrations of CoQ10 were achieved in blood, spleen, pancreas, liver, lungs, and in brain.

### Example 2. IV vs. Oral Administration of BPM31510

Patients were administered intravenously via infusion over 4 hours with BPM31510 at a dose of about 5.6 to 139 mg/kg. Separately, healthy volunteers were given BPM31510 orally at a daily dose of about 3,200 mg. Circulating (plasma) levels of CoQ10 in the oxidized form in the subjects were determined using a mass spectrometry based method as described herein and as described in PCT/US2013/028764, the entire contents of which are incorporated herein by reference.

As shown in table below, IV administration of BPM31510 resulted in an about 10000-fold higher CoQ10 exposure when compared to oral administration of BM31510 (i.e., Cmax of 2,424 μg/mL vs. 285 ng/mL, respectively). These data provide strong evidence for the use of BPM31510 via IV administration for treating CoQ10 deficiency. Oral administration of BPM31510 also resulted in plasma concentrations of CoQ10 greater than normal baseline concentrations found in normal healthy subjects.

| | BPM31510 (IV) | BPM31510 (PO) |
|---|---|---|
| Administration | IV (4 hours) | PO (TID) |
| Subjects | Patients | Healthy Volunteers |
| Dose | 5.6-139 mg/kg | 3,200 mg (~40 mg/kg/d) |
| Tmax (hours) | 4 | 2-4 |
| Cmax | 2,424 +/− 762 μg/mL | 285 +/− 254 ng/mL (SS) |
| | (for 139 mg/kg dose) | |
| AUC 0-4 (ng · hr/mL) | — | 905 +/− 464 |
| AUC 0-t (μg · hr/mL) | 40,660 +/− 17,443 | — |
| | (for 139 mg/kg dose) | |

### Example 3. In Vitro Evaluation of BPM31510

Fibroblasts from patients having primary CoQ10 deficiency are isolated and cultured in medium. Fibroblasts from healthy individuals are isolated and cultured as a control. Cells are treated with BPM31510, or soluble CoQ10 analogs for a certain amount of time. ATP production and relative oxygen species production are measured.

It is expected that cells treated with BPM31510 will have a corrected mitochondrial function as compared to untreated cells. It is also expected that the ATP production and relative oxygen species levels in the treated cells will be restored or be similar to the levels of ATP and relative oxygen species in fibroblast from healthy individuals, reflecting restoration of CoQ10 levels and CoQ10 activity in the fibroblasts from the patients . . .

### Example 4. In Vivo Efficacy of BPM31510 in Animal Models of Primary CoQ Deficiency

In order to evaluate the efficacy of BPM31510 in treating primary CoQ deficiency, translational models of primary CoQ deficiency (e.g., mice/rats/rodents having knocked-down expression levels of, or knock-out mutations in, the homologs of any one or more of Pdss1, Pdss2, Coq2, Coq4, Coq5, Coq6, Coq7, Coq8a/Adck3, Coq8b/Adck4, or Coq9) are treated with BPM31510 parenterally (e.g., intravenously), or optionally with vehicle control. Tissue (e.g., blood plasma, kidney, heart, brain, cerebellum, liver, and skeletal muscle) samples are collected to determine the levels of CoQ and compare the CoQ10 levels to the vehicle treated controls or to untreated animals.

It is expected that the plasma CoQ10 concentration in the animals administered the CoQ10 composition will have CoQ10 plasma concentrations greater than those of untreated or vehicle treated controls. It is further expected that the plasma CoQ10 concentration in the treated animals will approach, be similar to, or exceed the plasma CoQ10 concentration in healthy, wild type animals.

### Example 5. Efficacy of BPM31510 In Humans with Primary CoQ10 deficiency

Patients having primary CoQ10 deficiency (age>2 years) are selected based on genetic testing, and treated parenterally (e.g., intravenously) with BPM31510 at ascending doses. For example, the composition is administered at a daily dose of 5 mg/kg at Day 1, 10 mg/kg at Day 3, 15 mg/kg at Day 5, 20 mg/kg at Day 7, 25 mg/kg at Day 10, 50 mg/kg at Day 14, and 50 mg/kg at Day 24+.

Clinical scores, neurological qualify of life measures, brain function index (BFI), clinical measurements, CoQ10 plasma concentrations, and other biomarkers are evaluated on selected days (Day 1, Day 7, Day 14 and Day 24+) after dosing.

It is expected that administration of the CoQ10 composition to the patients will result in a significant increase in the patient's plasma CoQ10 concentration over the patient's baseline concentration (i.e., before the CoQ10 composition was administered), and that the plasma concentration will approach, be similar to, or exceed the plasma CoQ10 concentration in a healthy subject.

### Example 6. Preclinical Biodistribution of BPM31510

This example provides the preclinical in vivo biodistribution of BPM31510. Deficiency in CoQ10 exhibits pathophysiological effects on several tissues including brain (predominantly cerebellum effecting presentation of ataxia), kidney, heart, eye/ears, and muscle. To this extent, oxidized CoQ10 was evaluated in mice after 2 weeks of dosing with either a commercially available oral formulation (dosed orally) or BPM31510-IV (dosed intraperitoneally (IP)) at 10 mg/kg twice a day (BID) and 50 mg/kg BID. Tissues were harvested 4 hours after the last dosing. Control mice were dosed BID either orally or IP with vehicle (PBS). Increases in oxidized CoQ10 levels were not detectable in the tissues of the mice given oral CoQ10. Only IP dosing of BPM31510IV exhibited a dose dependent increase in oxidized CoQ10 concentration in susceptible tissues.

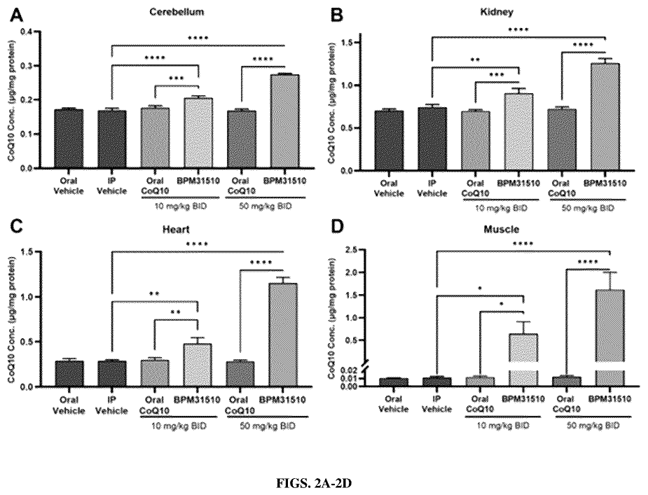

Cerebellum of BPM31510-IV treated mice showed a 1.2-fold increase (p<0.0001) in oxidized CoQ10 at 10 mg/kg BID and a 1.6-fold increase (p<0.0001) in oxidized CoQ10 at 50 mg/kg BID compared to vehicle and oral CoQ10 treated (FIG. 2A).

Kidney of BPM31510-IV treated mice showed a 1.2-fold increase (p=0.0033) in oxidized CoQ10 at the 10 mg/kg BID and a 1.7-fold increase (p<0.0001) in oxidized CoQ10 at the 50 mg/kg BID dose levels compared to vehicle and oral CoQ10 treated (FIG. 2B).

Heart tissue of BPM31510-IV treated mice showed a 1.7-fold increase (p=0.0018) in oxidized CoQ10 at 10 mg/kg BID and a 4.0-fold increase (p<0.0001) oxidized CoQ10 at 50 mg/kg BID dose levels compared to vehicle and oral CoQ10 treated (FIG. 2C).

Muscle of BPM31510-IV treated mice showed a 58-fold increase (p=0.0261) in oxidized CoQ10 at 10 mg/kg BID and a 147-fold increase (p<0.0001) oxidized CoQ10 at 50 mg/kg BID dose levels compared to vehicle and oral CoQ10 treated (FIG. 2D).

These data suggest that the lack of tissue CoQ10 is the potential reason for failure of oral supplementation to have significant efficacy after therapy, and also demonstrate the potential for BPM31510-IV to improve availability of CoQ10 in various tissues, e.g., CNS, kidney, heart, and muscle.

### Example 7. In Vitro Efficacy of BPM31510 in PABA-induced CoQ10 Deficient Neuronal Cells

An established model of CoQ10 deficiency was used to study the effects of BPM31510 in vitro. Para-aminobenzoic acid (PABA) has previously been reported to inhibit COQ2 activity thus inducing a CoQ10 deficiency in HL-60 cells. A neuronal cell model of CoQ10 deficiency by treatment of the neuronal SH-SY5Y cell line with PABA has previously been established. In this model, a significant reduction of cellular ATP production and increase in mitochondrial reactive oxygen species (ROS) was observed. Furthermore, CoQ10 supplementation (2.5 μM and 5 μM) was effective at significantly reducing mitochondrial oxidative stress. However, CoQ10 supplementation at these concentrations was unable to fully restore electron transport chain (ETC) activities (a treatment with >10 μM exogenous CoQ10 appeared be required to restore ETC activities). Therefore, this PABA-induced CoQ10 deficient in vitro model was used to evaluate the potency of BPM31510 to restore ATP levels and reduce mitochondrial ROS.

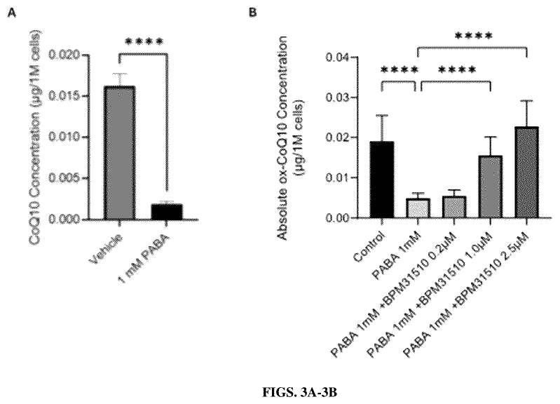

SH-SY5Y cells were pretreated for 5 days with 1 mM PABA or vehicle in all experiments. PABA treatment reduced oxidized CoQ10 levels by approximately 90% (FIG. 3A). Addition of 1 μM of BPM31510, for 5 days, to SH-SY5Y cells treated with 1 mM PABA, was sufficient to restore oxidized CoQ10 levels (FIG. 3B). No significant changes in cell viability, measured by CellTiter-Fluor, were observed between BPM31510 and vehicle control.

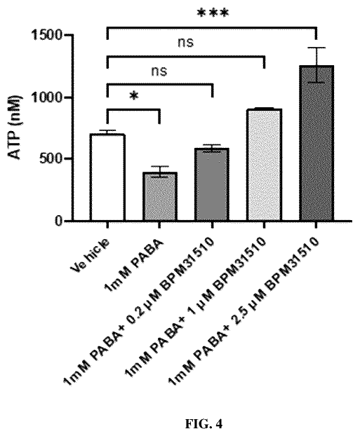

The decreased ATP levels induced by PABA treatment were restored with 0.2 μM treatment of BPM31510, while 2.5 μM resulted in a significant increase in ATP content above control values showing better than normal correction (FIG. 4).

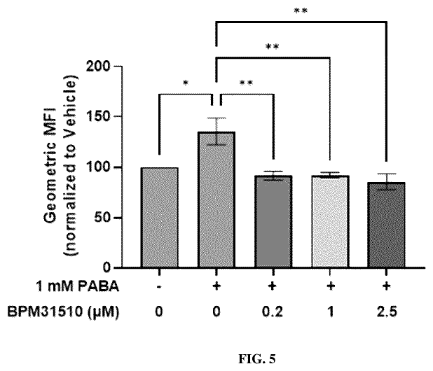

The PABA induced increase in mitochondrial ROS production, as measured by MitoSOX staining, was completely restored to control levels following 5-day treatment with 0.2 μM BPM31510 (FIG. 5).

These data together demonstrate that at concentrations as low as 0.2 μM (well below full correction of CoQ10 levels in cells), BPM31510 treatment was able to partially correct ATP levels and completely abrogate mitochondrial ROS production in CoQ10 deficient neuronal cells (PABA treated cells). At concentrations of luM, a full correction of CoQ10, ATP and mitochondrial ROS levels was observed in PABA treated cells. These findings contrast with reports in literature where correction of ATP and ROS levels are not complete, even with higher concentrations of unformulated CoQ10 (>10 μM). The results strongly support that BPM31510 is highly effective in restoring mitochondrial dysfunction (ATP production and mitochondrial ROS generation) induced by CoQ10 deficiency.

### Example 8. In Vitro Evaluation of BPM31510 in Fibroblasts from Patients with Primary CoQ10 Deficiency

Fibroblasts from patients having primary CoQ10 deficiency were isolated and cultured in medium. Cells were treated with BPM31510 or a control for 24 hours. ATP production levels were measured.

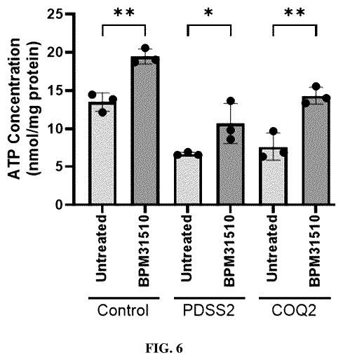

Measurement of ATP levels in CoQ10 deficient patient fibroblasts demonstrated that treatment with 5 μM BPM31510 for 24 hours increased ATP generation in PDSS2 and CoQ2 mutant fibroblast cell lines, restoring mitochondrial function (FIG. 6). These data demonstrate the pre-clinical efficacy of BPM31510.

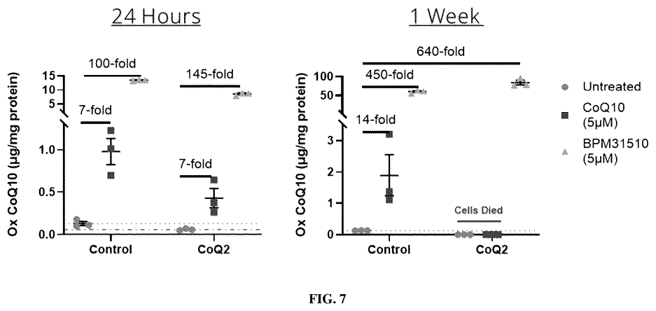

A further analysis was carried out comparing control fibroblast cell lines relative to CoQ2 deficient cell lines treated with BPM31510 vs a control CoQ10 alone for 24 hours and 1 week in galactose media. The results revealed that BPM31510 significantly increased CoQ10 content in cells over the time course (FIG. 7). This further impacted the survival of the CoQ2 deficient cell line after 1 week of treatment, since an equivalent dose of the control CoQ10 alone was not able to preserve survival of these cells, while BPM31510 was able to, due to its greater delivery to the cell.

### Example 9. Restoration of CoQ10 in Tissues of Primary CoQ10 Deficiency Mice (CoQ4 Mutation) Treated with BPM31510

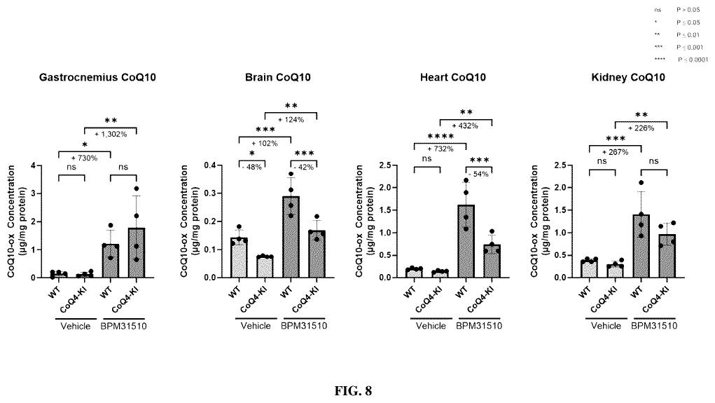

Primary CoQ10 deficiency mice having CoQ4 mutations were obtained. The CoQ4 knockin (KI) mice carried a point mutation in exon 5 of CoQ4 gene, c [440T>G], p.F147C, on C57BL/6 background. The point mutation is a homologue to the human mutation 437T>G; F146C. CoQ4 KI deficient mice were treated with BPM31510 or a vehicle control, with an IP dose of 200 mg/kg, every other day for two weeks. Wild type mice were also treated with BPM31510 or a vehicle control. Gastrocnemius muscle, brain, heart, and kidney tissue samples were collected and the levels of CoQ10 were measured by an absolute quantitative LC MS/MS method. As shown in FIG. 8, Treatment of CoQ4 KI deficient mice with BPM31510 resulted in a significant increase in the CoQ10 levels in gastrocnemius muscle, brain, heart, and kidney tissues, restoring CoQ levels in these tissues in the deficient mouse model.

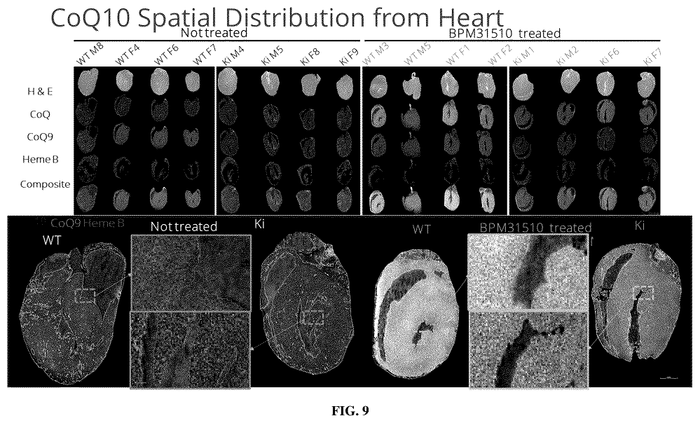

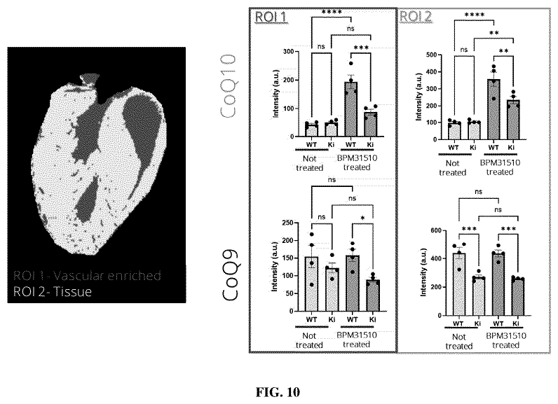

Spatial omics analysis of CoQ4 KI deficient mice revealed a deficiency of CoQ9 in the heart of this mouse model and that CoQ10 was absorbed in the myocardium of the heart. This imaging technique clearly demonstrates that CoQ10 is enriched in the endothelial vessel wall in both WT and CoQ4 KI mutant mice as well through cardiomyocytes (FIG. 9). Utilizing a mass spec imaging segmentation technique isolating vascular enriched locations (ROI 1) due to the localization of Heme b vs muscle tissue enriched regions (ROI 2), CoQ10 statically increased in CoQ4 KI mutant mice increasing the overall CoQ pool (FIG. 10).

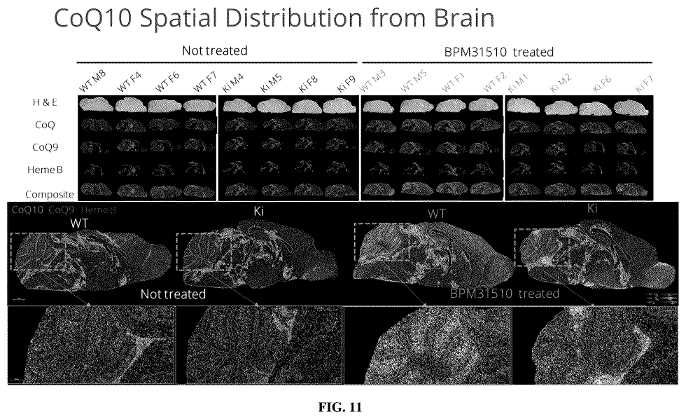

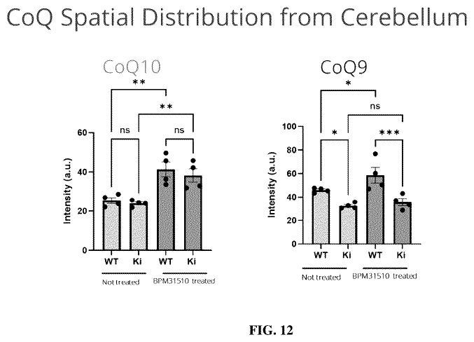

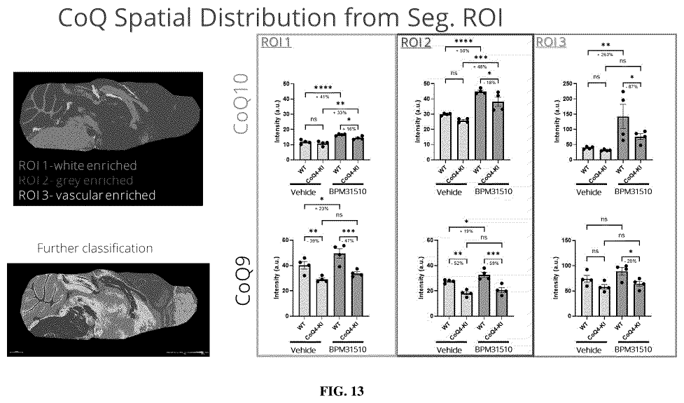

Spatial omics analysis of brain regions demonstrated a clear decrease in CoQ9 in the CoQ4 KI mice and that CoQ10 was able to reach the brain spatially across the brain regions in BPM31510 treated animals (FIG. 11). Notably, CoQ10 was able to reach the cerebellum, which is critical for the ataxic phenotype in CoQ10 deficient patients. Quantitation of CoQ10 using imaging techniques with deuterated CoQ10 standard sprayed on the tissue sample for pixel normalization revealed a statistical increase of CoQ10 in BPM31510 treated WT mice and CoQ4 KI mice, demonstrating a restoration of the CoQ pool in the cerebellum over the course of two weeks of treatment (FIG. 12). Utilizing segmentation analysis, it evidenced that CoQ10 increased in white matter enriched regions (RO1) and grey matter enriched regions (RO2) in BPM31510 treated animals (FIG. 13). CoQ9 was not changed in the treated mice, although the overall pool of CoQ was increased.

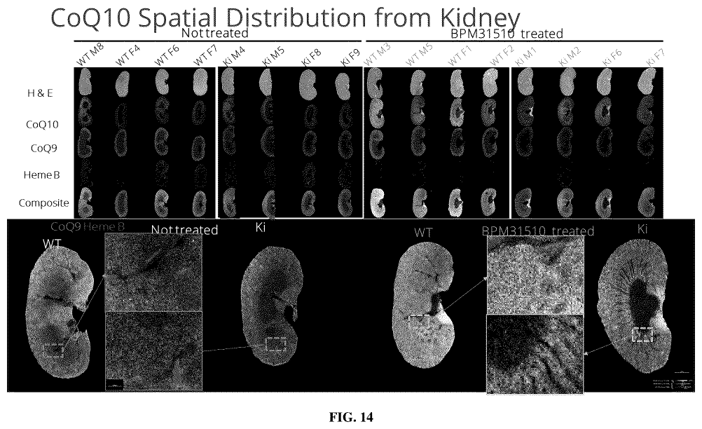

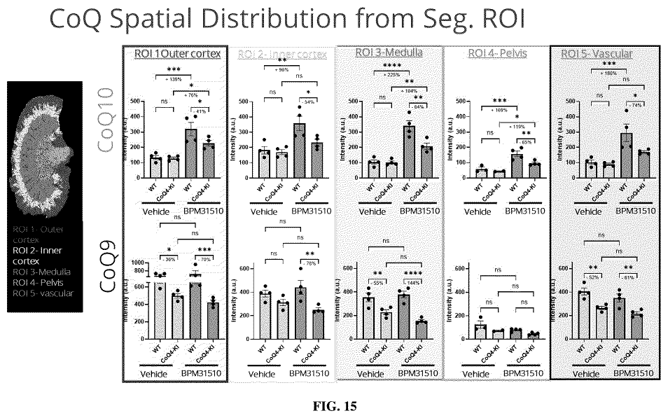

Spatial omics analysis was also performed for the kidney regions. As shown in FIG. 14, CoQ10 was able to reach the kidney in BPM31510 treated animals. Quantitation of CoQ10 using imaging techniques with deuterated CoQ10 standard sprayed on the kidney tissue sample for pixel normalization revealed a statistical increase in CoQ10 in BPM31510 treated WT and CoQ4 KI mice in the outer cortex, medulla, and pelvis of the kidney over the course of two weeks of treatment (FIG. 15).

### Example 10. Efficacy of BPM31510 in Humans with Primary CoQ10 Deficiency

Two patients having primary CoQ10 deficiency (CoQ8A mutations) were selected based on genetic testing as shown in Table 1, and treated parenterally (intravenously) with BPM31510 at ascending doses. Specifically, BPM31510 was administered via IV dosing over 4 hours at a daily dose of 10 mg/kg on Day 1, 20 mg/kg on Day 8, 40 mg/kg on Day 15, and 50 mg/kg on Day 22+. Patients also received 30 mg/kg of a commercially available oral CoQ10 formulation while on BPM31510 treatment.

Clinical scores, physical global assessment of changes, clinical measurements, and relevant biomarkers were evaluated on Day 1, Day 8, Day 15 and Day 22+after doing. Table 1 provides the relevant characteristics of patients receiving the BPM31510 treatment.

TABLE 1
Patient Characteristics

| Patients | COQ01 | COQ02 |
|---|---|---|
| COQ8A mutation(s) | c.1042C > T; | c.812G > A; |
| | p.Arg348* | p.Arg271His |
| | (homozygous) | c.1821C > A; |
| | | p.Tyr607Ter |
| Sex | Male | Female |
| Age of Onset (years) | <=5 (motor development) | 2 (motor development) |
| Clinical Syndrome | Cerebellar ataxia, | Cerebellar ataxia, |
| | tremor, mild cognitive | mild cognitive |
| | deficits | deficits |
| Current Age (years) | 16 | 9 |
| SARA at baseline ′24 | 13 | 11 |
| Oral CoQ₁₀ | 30 mg/kg | 30 mg/kg (sublingual) |

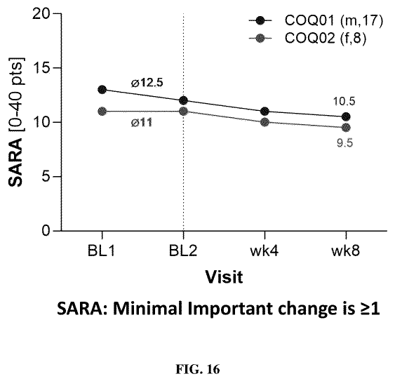

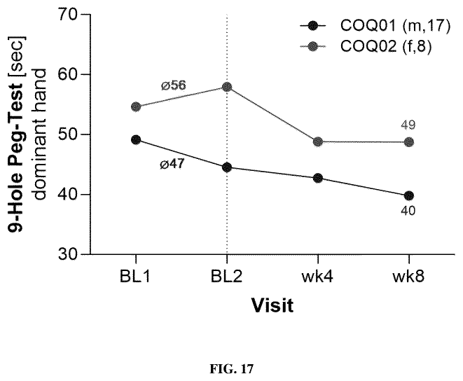

As shown in FIGS. 16-17, patients had a highly significant improvement in the clinical-reported outcome (Scale for the Assessment and Rating of Ataxia (SARA) score) and the performance outcome (9-Hole Peg Test (9HPT)).

SARA is a clinical scale which assesses a range of different impairments in cerebellar ataxia. The scale is made up of 8 items related to gait, stance, sitting, speech, finger-chase test, nose-finger test, fast alternating movements and heel-shin test with accumulative score ranging from 0 (no ataxia) to 40 (most severe ataxia). A minimal important change in SARA score is ≥1. As shown in FIG. 16, both patients had a highly significant improvement in the SARA score of about 1.5 and 2, respectively.

The 9HPT is used to measure finger dexterity in patients with various neurological diagnoses. A significant improvement of greater than 5 seconds was observed for both patients (FIG. 17).

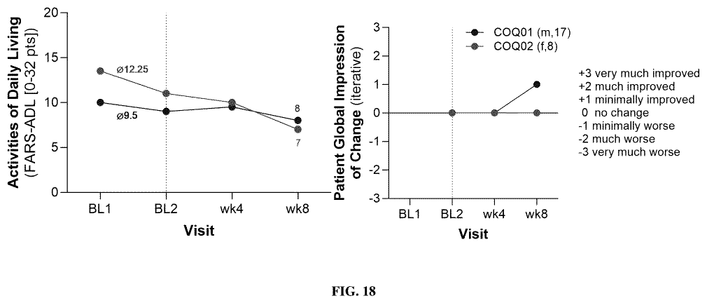

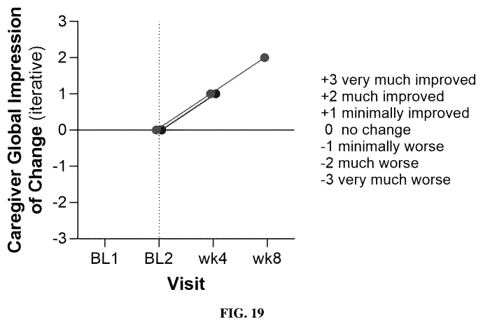

Similarly, a significant improvement was observed for both patient-reported outcomes (Friedreich's Ataxia Rating Scale-Activities of Daily Living (FARS-ADL) and Patient Global Impression of Change (PGIC)) (FIG. 18) and observer-reported outcomes (FIG. 19). FARS-ADL and PGIC are both used to measure the severity of ataxia and how it affects a patient's daily life. FARS-ADL is a scale that assesses a patient's ability to perform daily tasks, while PGIC is a measure of how a patient perceives their own change in condition.

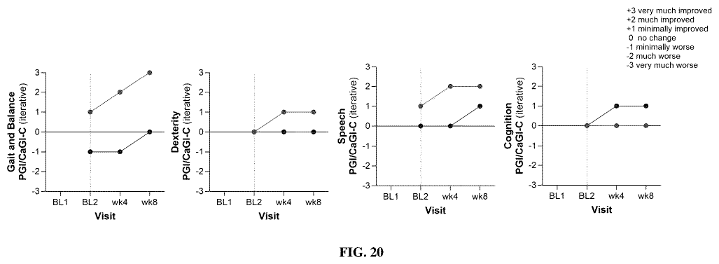

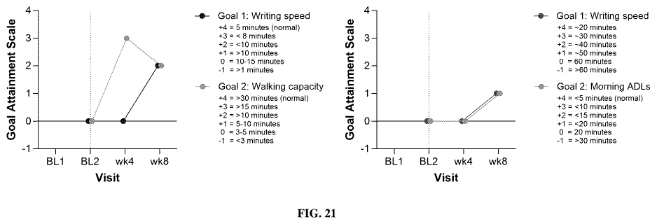

Improvement was also observed with respect to gait and balance, dexterity, speech and cognition (FIG. 20), as well as goal attainment (FIG. 21). No side effects, particularly no bleeding events, and no laboratory changes, such as coagulation parameters, were observed.

These data demonstrate that administration of BPM31510 to the patients result in a significant increase in all clinical outcomes measured, and provide strong evidence for the use of BPM31510 via IV administration for treating CoQ10 deficiency.

### Example 11. Efficacy of BPM31510 in Humans with Primary CoQ10 Deficiency

A phase 3 clinical trial study is designed to evaluate the efficacy of BPM31510 in patients having primary CoQ10 deficiency. Patients having primary CoQ10 deficiency (age>2 years) are selected based on genetic testing, and treated parenterally (e.g., intravenously) with BPM31510 at a dose of 50 mg/kg weekly.

The duration of the study is about 12-24 weeks, followed by an open label extension (OLE) study of 48 weeks. Patients are screened for clinical assessments at each weekly visit. The primary endpoint is PROMIS fatigue and mobility score. Secondary endpoints include 6MWT, 9-HPT, proteinuria, neurological quality of life assessment, caregiver quality of life assessment, pharmacokinetics, biomarkers such as lactate, pyruvate, FGF21 and GDF15, and fatigue level.

It is expected that patients receiving BPM31510 will have a significant increase in the patient's plasma CoQ10 concentration over the patient's baseline concentration (i.e., before the CoQ10 composition was administered), and that the plasma concentration will approach, be similar to, or exceed the plasma CoQ10 concentration in a healthy subject. In addition, it is expected that patients receiving BPM31510 will have a significant change in the PROMIS fatigue and mobility score and the secondary endpoints.

1. A method for treating primary Coenzyme Q10 (CoQ10) deficiency in a subject in need thereof, comprising administering parenterally to the subject a therapeutically effective amount of a composition comprising CoQ10, thereby treating the subject.

2. The method of claim 1, wherein a plasma concentration of at least about 10 μg/mL or 0.01 mM for Co10 is achieved in the subject.

3. A method for treating primary Coenzyme Q10 (CoQ10) deficiency in a subject in need thereof, comprising (i) administering parenterally to the subject a therapeutically effective amount of a composition comprising CoQ10, and (ii) administering orally a therapeutically effective amount of the composition comprising CoQ10; such that a plasma concentration of at least about 10 μg/mL or 0.01 mM for Co10 is achieved in the subject, thereby treating the subject.

4. The method of claim 1, further comprising selecting a subject determined as having primary CoQ10 deficiency.

5. The method of claim 4, wherein the subject is determined as having primary CoQ10 deficiency based on the presence of a loss of function in one or more genes selected from the group consisting of PDSS1, PDSS2, COQ2, COQ4, COQ5, COQ6, COQ7, COQ8A, COQ8B, and COQ9.

6. The method of claim 1,
(a) wherein a plasma concentration of about 10 μg/mL to about 3000 μg/mL, or about 0.01 mM to about 3.0 mM, for CoQ10 is achieved in the subject;
(b) wherein a plasma concentration of about 10 μg/mL to about 2500 μg/mL, or about 0.01 mM to about 2.5 mM, for CoQ10 is achieved in the subject;
(c) wherein a plasma concentration of about 10 μg/mL to about 2000 μg/mL, or about 0.01 mM to about 2.0 mM, for CoQ10 is achieved in the subject;
(d) wherein a plasma concentration of at least about 20 μg/mL or 0.02 mM for CoQ10 is achieved in the subject;
(e) wherein a plasma concentration of at least about 50 μg/mL or 0.05 mM for CoQ10 is achieved in the subject;
(f) wherein a plasma concentration of at least about 100 μg/mL or 0.1 mM for CoQ10 is achieved in the subject;
(g) wherein a plasma concentration of at least about 200 μg/mL or 0.2 mM for CoQ10 is achieved in the subject; and/or
(h) wherein a plasma concentration of at least about 500 μg/mL or 0.5 mM for CoQ10 is achieved in the subject.

7-13. (canceled)

14. The method of claim 1,
(a) wherein the composition is formulated as a nano-dispersion; and/or
(b) wherein the composition comprises an aqueous solution; a CoQ10 dispersed into a colloidal nano-dispersion of particles; and at least one of a dispersion stabilizing agent and an opsonization reducer; wherein the nano-dispersion of the CoQ10 is dispersed into nano-particles having a mean particle size of less than 200-nm.

15. (canceled)

16. The method of claim 14,
(a) wherein the dispersion stabilizing agent is selected the group consisting of pegylated castor oil, Cremphor EL, Cremophor RH 40, Pegylated vitamin E, Vitamin E TPGS, and Dimyristoylphosphatidyl choline (DMPC);
(b) wherein the dispersion stabilizing agent is DMPC;
(c) wherein the oposonization reducer is selected from the group consisting of poloxamer and poloxamines;
(d) wherein the opsonization reducer is poloxamer 188;
(e) wherein the opsonization reducer is poloxamer 188 and the dispersion stabilizing agent is DMPC;
(f) wherein the colloidal nano-dispersion is a suspension;
(g) wherein the colloidal nano-dispersion is an emulsion;
(h) wherein the CoQ10 of the colloidal nano-dispersion is in a crystalline form;
(i) wherein the CoQ10 of the colloidal nano-dispersion is in a super-cooled melt form;
(j) wherein the composition comprising CoQ10 has a weight-per-volume of the CoQ10, DMPC and poloxamer of 4%, 3% and 1.5%, respectively;
(k) wherein the composition comprising CoQ10 has a weight-per-volume of the CoQ10, DMPC and poloxamer 8%, 6% and 3%, respectively;
(l) wherein the size of the nano-dispersion particles is between 10-nm and 200-nm;
(m) wherein the size of the nano-dispersion particles is between 10-nm and 100-nm; and/or
(n) wherein the size of the nano-dispersion particles is between 35-nm and 40-nm.

17-29. (canceled)

30. A method for treating primary Coenzyme Q10 (CoQ10) deficiency in a subject in need thereof, comprising administering parenterally to the subject a therapeutically effective amount of a composition comprising CoQ10,
wherein the composition comprises an aqueous solution; a CoQ10 dispersed into a colloidal nano-dispersion of particles; and at least one of a dispersion stabilizing agent and an opsonization reducer; wherein the nano-dispersion of the CoQ10 is dispersed into nano-particles having a mean particle size of less than 200-nm, thereby treating the subject.

31. The method of claim 30, further comprising administering orally a therapeutically effective amount of the composition comprising CoQ10.

32. The method of claim 30- or 31, further comprising selecting a subject determined as having primary CoQ10 deficiency.

33. The method of claim 32, wherein the subject is determined as having primary CoQ10 deficiency based on the presence of a loss of function in one or more genes selected from the group consisting of PDSS1, PDSS2, COQ2, COQ4, COQ5, COQ6, COQ7, COQ8A, COQ8B, and COQ9.

34. The method of claim 30,
(a) wherein a plasma concentration of about 10 μg/mL to about 3000 μg/mL, or about 0.01 mM to about 3.0 mM, for CoQ10 is achieved in the subject;
(b) wherein a plasma concentration of about 10 μg/mL to about 2500 μg/mL, or about 0.01 mM to about 2.5 mM, for CoQ10 is achieved in the subject;
(c) wherein a plasma concentration of about 10 μg/mL to about 2000 μg/mL, or about 0.01 mM to about 2.0 mM, for CoQ10 is achieved in the subject;
(d) wherein a plasma concentration of at least about 10 μg/mL or 0.01 mM for CoQ10 is achieved in the subject;
(e) wherein a plasma concentration of at least about 20 μg/mL or 0.02 mM for CoQ10 is achieved in the subject;
(f) wherein a plasma concentration of at least about 50 μg/mL or 0.05 mM for CoQ10 is achieved in the subject;
(g) wherein a plasma concentration of at least about 100 μg/mL or 0.1 mM for CoQ10 is achieved in the subject;
(h) wherein a plasma concentration of at least about 200 μg/mL or 0.2 mM for CoQ10 is achieved in the subject; and/or
(i) wherein a plasma concentration of at least about 500 μg/mL or 0.5 mM for CoQ10 is achieved in the subject.

35-42. (canceled)

43. The method of claim 30- or 31,
(a) wherein the dispersion stabilizing agent is selected the group consisting of pegylated castor oil, Cremphor EL, Cremophor RH 40, Pegylated vitamin E, Vitamin E TPGS, and Dimyristoylphosphatidyl choline (DMPC);
(b) wherein the dispersion stabilizing agent is DMPC;
(c) wherein the oposonization reducer is selected from the group consisting of poloxamer and poloxamines;
(d) wherein the opsonization reducer is poloxamer 188;
(e) wherein the opsonization reducer is poloxamer 188 and the dispersion stabilizing agent is DMPC;
(f) wherein the colloidal nano-dispersion is a suspension;
(g) wherein the colloidal nano-dispersion is an emulsion;
(h) wherein the CoQ10 of the colloidal nano-dispersion is in a crystalline form;
(i) wherein the CoQ10 of the colloidal nano-dispersion is in a super-cooled melt form;
(j) wherein the composition comprising CoQ10 has a weight-per-volume of the CoQ10, DMPC and poloxamer of 4%, 3% and 1.5%, respectively;
(k) wherein the composition comprising CoQ10 has a weight-per-volume of the CoQ10, DMPC and poloxamer 8%, 6% and 3%, respectively;
(l) wherein the size of the nano-dispersion particles is between 10-nm and 200-nm;
(m) wherein the size of the nano-dispersion particles is between 10-nm and 100-nm; and/or
(n) wherein the size of the nano-dispersion particles is between 35-nm and 40-nm.

44-56. (canceled)

57. The method of claim 1,
(a) wherein the composition is administered by intravenous infusion;
(b) wherein the composition is administered intravenously once per week;
(c) wherein the composition is administered intravenously twice per week;
(d) wherein the composition is administered intravenously at a dose of between about 5 mg/kg to about 350 mg/kg of CoQ10 per week;
(e) wherein the composition is administered intravenously at a dose of between about 10 mg/kg to about 100 mg/kg of CoQ10 per week;
(f) wherein the composition is administered intravenously at a dose of between about 25 mg/kg to about 75 mg/kg of CoQ10 per week;
(g) wherein the composition is administered intravenously at a dose of about 5 mg/kg, about 10 mg/kg, about 15 mg/kg, about 20 mg/kg, about 25 mg/kg, about 50 mg/kg, about 100 mg/kg, about 150 mg/kg, about 200 mg/kg, about 250 mg/kg, about 300 mg/kg, or about 350 mg/kg of CoQ10 per week; and/or
(h) wherein the composition is administered intravenously at a dose of about 50 mg/kg of CoQ10 per week.

58-64. (canceled)

65. The method of claim 3,
(a) wherein the composition is administered orally according to an administration schedule selected from once per week, twice per week, three times per week, four times per week, five times per week, six times per week, or once per day;
(b) wherein the composition is administered orally at a dose of between about 5 mg/kg to about 5000 mg/kg of CoQ10 per day;
(c) wherein the composition is administered orally at a dose of between about 10 mg/kg to about 3500 mg/kg of CoQ10 per day;
(d) wherein the composition is administered orally at a dose of between about 20 mg/kg to about 3500 mg/kg of CoQ10 per day; and/or
(e) wherein the composition is administered orally at a dose of about 5 mg/kg, about 10 mg/kg, about 15 mg/kg, about 20 mg/kg, about 25 mg/kg, about 50 mg/kg, about 100 mg/kg about 200 mg/kg, about 500 mg/kg, about 1000 mg/kg, about 1500 mg/kg, about 2000 mg/kg, about 2500 mg/kg, about 3000 mg/kg, or about 3500 mg/kg of CoQ10 per day.

66-69. (canceled)

70. The method of claim 1, further comprising administering a CoQ10 supplement orally to the subject.

71. The method of claim 70, wherein the CoQ10 supplement is administered orally at a dose of about 10 to about 100 mg/kg per day.

72. The method of claim 1, wherein the subject is a human subject.

73. The method of claim 1,
(a) wherein administration of the composition comprising CoQ10 results in a concentration of CoQ10 in the brain, heart, kidney, and/or muscle tissues that is at least 1.5-fold greater than the concentration of CoQ10 in the brain, heart, kidney, and/or muscle tissues measured in the subject prior to administration of the composition;
(b) wherein administration of the composition comprising CoQ10 restores the level of CoQ10 in the subject to a normal physiological level or above the physiological level in the brain, heart, kidney and/or muscle tissues of the subject;
(c) wherein administration of the composition comprising CoQ10 results in a change in the levels of lactate, succinate and/or citrate in the brain, heart, kidney and/or muscle tissues of the subject;
(d) wherein administration of the composition comprising CoQ10 increases the plasma concentration of one or more metabolites of quinone metabolism, optionally,
wherein the one or more metabolites of quinone metabolism is selected from the group consisting of Coenzyme Q1, Coenzyme Q2, Coenzyme Q4, phylloquinone, menaquinone, menadione, 1,2-naphthoquinone, D-alpha-tocopherylquinone, p-Benzoquinone, duroquinone, idebenone, 2-methoxy-1,4-naphthoquinone, 2,6-dimethoxy-1,4-benzoquinone, adrenochrome, 1,8-dihydroxyanthraquinone, chrysophanol, thymoquinone, 2,6-di-tert-butyl-1,4-benzoquinone, emodin, and pyrroloquinoline quinone;
(e) wherein administration of the composition comprising CoQ10 results in a decrease in a (Scale for the Assessment and Rating of Ataxia (SARA) score by at least 1, 1.1, 1.2, 1.3, 1.4, 1.5, 1.6, 1.7, 1.8, 1.9, 2, 2.5, or 3, as compared to a SARA score measured in the subject prior to administration of the composition;
(f) wherein administration of the composition comprising CoQ10 results in a decrease in the time required to complete a 9-Hole Peg Test (9HPT) by at least 1, 2, 3, 4, 5, 6, 7, 8, 9, or 10 seconds, as compared to the time required to complete the test by the subject prior to administration of the composition;
(g) wherein administration of the composition comprising CoQ10 results in a decrease in a Friedreich's Ataxia Rating Scale-Activities of Daily Living (FARS-ADL) score by at least 1, 1.5, 2, 2.5, 3, 3.5, 4, 4.5, 5, 5.5, or 6, as compared to a FARS-ADL score measured in the subject prior to administration of the composition; and/or
(h) wherein administration of the composition comprising CoQ10 results in an increase in a Patient Global Impression of Change (PGIC) score by at least 1, 2, or 3, as compared to a PGIC score measured in the subject prior to administration of the composition.

74-82. (canceled)
